(19) United States
(12) Patent Application Publication
(10) Pub. No.: US 2026/0242492 A1
(43) Pub. Date: Aug. 20, 2026

(54) ANTIBODY-LIKE MOLECULE COMPRISING HETERODIMER OF HUMAN CD1B (CLUSTER OF DIFFERENTIATION 1) PROTEIN

(71) Applicant: JOINT STOCK COMPANY "BIOCAD", Saint Petersburg (RU)

(72) Inventors: Dmitrii Nikolaevich POLIAKOV, Saint Petersburg (RU); Sergei Aleksandrovich LEGOTSKII, Moscow (RU); Maksim Andreevich DANILOV, Saint Petersburg (RU); Marianna Dmitrievna BARANOVSKAIA, Saint Petersburg (RU); Kseniia Vitalievna IVANOVA, Saint Petersburg (RU); Olga Viktorovna NAZARENKO, gp Kuzmolovskij (RU); Pavel Andreevich IAKOVLEV, Saint Petersburg (RU); Dmitry Valentinovich MOROZOV, Saint Petersburg (RU)

(21) Appl. No.: 19/162,218

(22) PCT Filed: Feb. 27, 2024
(86) PCT No.: PCT/RU2024/050045

§ 371 Date: Sep. 4, 2025

(30) Foreign Application Priority Data
Mar. 6, 2023 (RU) 2023105130

(51) Int. Cl. C07K 16/28; C07K 14/705

(52) U.S. Cl. CPC C07K 16/2866; C07K 14/70539; C07K 16/2818; C07K 16/2887; C07K 2317/31; C07K 2317/522; C07K 2317/524; C07K 2317/526; C07K 2317/55; C07K 2317/92; C07K 2318/10; C07K 2319/30

(57) ABSTRACT

The present invention relates to the field of biotechnology, specifically to antibody-like molecules that comprise a heterodimer of membrane-proximal domains of human CD1b (cluster of differentiation 1) protein, as well as to a process for producing said antibody-like molecules. The invention further relates to a nucleic acid encoding said antibody-like molecule, an expression vector, a host cell for producing said antibody-like molecule and to a method for producing said cell.

### FIELD OF THE INVENTION

The present invention relates to the field of biotechnology, specifically to antibody-like molecules that comprise a heterodimer of membrane-proximal domains of human CD1b (cluster of differentiation 1) protein, as well as to a process for producing said antibody-like molecules. The invention further relates to a nucleic acid encoding said antibody-like molecule, an expression vector, a host cell for producing said antibody-like molecule and to a method for producing said cell.

### BACKGROUND OF THE INVENTION

Monoclonal antibodies in the form of chimeric, humanized or fully human molecules have proven to be useful as effective medicine for treating multiple disorders and diseases.

Natural human antibody molecules are represented by heterotetramers consisting of two light and two heavy chains: heavy chains form a homodimer and each heavy chain forms a heterodimer with a cognate light chain. Conventional monoclonal antibodies in the form of whole molecules consist of bivalent (“two-armed”) heterotetramers of heavy and light chains.

Diseases are often caused as a result of several pathologies and are accompanied by many concomitant diseases. Bispecific and multispecific antibodies are capable of binding and thereby neutralizing two or more distinct antigens per antibody molecule. The potential for a significant improvement in the therapeutic properties (and value) of medicinal products as compared to monospecific monoclonal antibodies has made bispecific and multispecific antibodies an active area of research. Over the past twenty years, the literature has described many solutions regarding engineered versions of bispecific antibodies, as described in Brinkmann, U and RE Kontermann, 2017, The Making of Bispecific Antibodies, MAbs; 209 February/March; 9(2):182-212, doi: 10.1080/19420862.2016.1268307.

As mentioned above, there are many approaches to create molecules with combined antigen-binding domains, i.e. with antigen-binding domains that differ from one another. However each of these methods has its disadvantages.

Cross-linking by chemical methods is a time-consuming process, since the corresponding portions should be purified from homodimers and other undesirable by-products. In addition, the steps of chemical modification may alter the integrity of proteins, thus impairing stability thereof. Thus, the above method is typically ineffective and may lead to the loss of antibody activity.

A method based on cell fusion (for example, production of hybridomas) is an arbitrary assembly of two heavy and two light chains, resulting in 10 combinations of antibodies. Target heteromultimeric antibodies are only a small portion of antibodies produced in this fashion. Isolation of target heteromultimeric proteins significantly reduces product yield and increases production costs.

Recombinant DNA techniques are employed to create various heteromultimeric antibodies, for example, single-chain Fv fragments, diabodies, etc. that are free of an Fc fragment. The main disadvantage of this type of an antibody molecule is an absent Fc domain, which results in antibody failure to trigger an effector function (such as, for example, complement activation, binding to Fc receptor, and so forth). Thus, there is a need for a bispecific or multispecific antibody comprising a functional Fc domain.

Recombinant DNA techniques are also employed to design bispecific or multispecific antibodies coupled using the Knob-into-Holes technology (see, for example, publications of international applications WO9627011 and WO9850431). One factor limiting the use of the above method is the fact that the light chains of the two initial antibodies should be identical to prevent mispairing and formation of undesirable and/or inactive molecules when expressed in a single cell.

The purity of bispecific or multispecific antibody product depends on two factors as follows:
a) heterodimeric assembly of two distinct heavy chains co-expressed in a cell, and
b) correct pairing of two distinct light chains to cognate heavy chains.

The “Knob-into-Holes” technology to design bispecific or multispecific antibodies solves the problem of correct heterodimeric assembly of two distinct heavy chains co-expressed in a cell. However, the use of only the Knob-into-Holes technology to design bispecific or multispecific antibodies makes it possible to achieve only about 25% yield of correctly assembled bispecific or multispecific product, since the problem of correct pairing of two distinct light chains to their cognate heavy chains remains unresolved.

The problem of correct pairing of two distinct light chains to their cognate heavy chains is solved in various fashions as follows:

1. Use of an identical light chain in first and second antigen-binding portions of antibody (Van Blarcom T ET AL., Productive common light chain libraries yield diverse panels of high affinity bispecific antibodies, MAbs. 2018 February/March; 10(2):256-268. doi: 10.1080/19420862.2017.1406570).

The disadvantage of the above solution is non-universality thereof, since it may be problematic to select a light chain suitable for the both valences. Furthermore, in case of amino acid substitutions in the light chain to optimize the properties of the antigen-binding fragment, the substitutions will affect the both valences. Further, antibody binding to the second antigen may be disrupted.

2. Use of single-chain formats, i.e. the formats where the light and heavy chains of the antigen-binding fragment specific for the first antigen are connected to one another via a linker of several amino acids.

This format has technological disadvantages, since it uses linkers either to fuse the antibody core (IgA, IgD, IgE, IgG or IgM) to a further binding protein (for example, scFv or scFab), or to fuse, for example, light and heavy variable domains (VH and VL) within scFv or a light chain (VL-CK(or CL)) to VH-CH1 within scFab. Linkers may cause problems in therapeutic settings. In fact, these foreign peptides can elicit an immune response against the linker itself or the junction region between the protein and the linker. Furthermore, the flexible nature of these peptides and the mobility thereof make them more prone to proteolytic cleavage, potentially leading to poor antibody stability, aggregation and increased immunogenicity.

3. Modification of CH1-CK domains in a bispecific or multispecific antibody to allow altering the interaction interface in bispecific or multispecific antibody expression techniques so as to exclude incorrect association of light chains. For example, the publication of international application WO2017059551 provides various amino acid substitutions in CH1 and/or CK that promote the preferred pairing between the desired heavy chain and the desired light chain.

Despite the above various bispecific or multispecific antibody expression technologies, there is still a need in the art for improved purity of the bispecific or multispecific antibody product, as well as for a scalable production solution for producing correctly assembled bispecific or multispecific antibodies.

### DISCLOSURE OF THE INVENTION

Developed by the authors of the present invention, the novel format of antibody-like molecules that comprise a heterodimer of membrane-proximal domains of CD1b (cluster of differentiation 1) protein, as well as the process for producing said antibody-like molecules surprisingly allow for producing a high yield of product with correct heterodimeric assembly of two distinct heavy chains co-expressed in a cell and with correct pairing of two distinct light chains to cognate heavy chains.

Developed by the authors of the present invention, the novel format of antibody-like molecules that comprise a heterodimer of membrane-proximal domains of the CD1b protein, as well as the process for producing said antibody-like molecules surprisingly allow for producing a correctly assembled product of antibody-like molecules with high purity.

Consequently, the above results reduce production costs and lead to a scalable production solution for producing properly assembled antibody-like molecules.

### Definitions and General Methods

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

The term “KD” in this description refers to the affinity constant (or equilibrium constant) which is calculated from the ratio of Kd to Ka (i.e. Kd/Ka), and it is expressed as a molar concentration (M).

“Binding affinity” typically refers to the strength of the sum total of noncovalent interactions between a single binding site of a molecule (e.g. an antibody) and its binding partner (e.g. an antigen). Unless indicated otherwise, “binding affinity” refers to intrinsic (characteristic, true) binding affinity which reflects a 1:1 interaction between members of a binding pair (e.g. antibody and antigen). Affinity of a molecule X for its binding partner Y can typically be represented by the affinity constant (KD). The preferred Kd value is about 200 nM, 150 nM, 100 nM, 60 nM, 50 nM, 40 nM, 30 nM, 20 nM, 10 nM, 8 nM, 6 nM, 4 nM, 2 nM, 1 nM, or less. Affinity can be measured by common methods known in the art, including those described in the present description. Low-affinity antibodies typically bind an antigen slowly and tend to dissociate readily, whereas high-affinity antibodies typically bind an antigen faster and tend to remain bound longer. A variety of methods for measuring binding affinity are known in the art, any one of these methods may be used for the purposes of the present invention.

The term “Kd”, “koff” or “kdis” refers to the off rate constant of a particular interaction between a binding molecule and antigen. The off rate constant koff can be measured using bio-layer interferometry, for example, using the Octet™ system.

The term “Ka”, “kon” or “on-rate” refers to the association rate constant.

The term “R²” refers to the coefficient of determination.

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

### Antibody-Like Molecule

The present invention relates to an antibody-like molecule that specifically binds to first and second targets.

The antibody-like molecule according to the invention is a monoclonal antibody-like molecule.

The term “monoclonal antibody-like molecule” refers to an antibody-like molecule that is synthesized and secreted by a separate clonal population of cells.

The antibody-like molecule according to the invention is a recombinant antibody-like molecule.

The term “recombinant antibody-like molecule” refers to an antibody-like molecule that is expressed in a cell or cell line comprising nucleotide sequence(s) encoding the antibody-like molecule, wherein said nucleotide sequence(s) is (are) not associated with the cell in nature.

The antibody-like molecule according to the invention is an isolated antibody-like molecule.

The term “isolated” used to describe various antibody-like molecules according to the present description refers to an antibody-like molecule which has been identified and isolated and/or regenerated from a cell or cell culture in which it is expressed. Impurities (contaminant components) from natural environment are materials which typically interfere with diagnostic or therapeutic uses of the polypeptide, and may include enzymes, hormones, and other proteinaceous or nonproteinaceous solutes. The isolated polypeptide is typically prepared by at least one purification step.

In one aspect, the present invention relates to an antibody-like molecule that specifically binds to first and second targets, wherein said antibody-like molecule comprises:
(i) a) a light chain constant domain that is β2 microglobulin (β2M) of the human CD1b protein; and
b) a first heavy chain constant domain that is the α3 membrane-proximal domain of the human CD1b protein;
or
(ii) a) a light chain constant domain that is the α3 membrane-proximal domain of the human CD1b protein;
and
b) a first heavy chain constant domain that is β2 microglobulin (β2M) of the human CD1b protein;
wherein the β2 microglobulin (β2M) of the human CD1b protein and the α3 membrane-proximal domain of the human CD1b protein form therebetween a heterodimer that is stabilized by a disulfide bond.

The α3 domain of the human CD1b protein (α3 membrane-proximal domain of the human CD1b protein) and β2 microglobulin of the human CD1b protein are membrane-proximal domains of the human CD1b protein.

In one aspect, the present invention relates to an antibody-like molecule that specifically binds to first and second targets, wherein said antibody-like molecule comprises:
(i) a) a light chain constant domain that is β2 microglobulin (β2M) of the human CD1b protein with the amino acid sequence of SEQ ID NO: 1;
and
b) a first heavy chain constant domain that is the α3 membrane-proximal domain of the human CD1b protein with the amino acid sequence of SEQ ID NO: 2;
or
(ii) a) a light chain constant domain that is the α3 membrane-proximal domain of the human CD1b protein with the amino acid sequence of SEQ ID NO: 2;
and
b) a first heavy chain constant domain that is β2 microglobulin (β2M) of the human CD1b protein with the amino acid sequence of SEQ ID NO: 1;
wherein the β2 microglobulin (β2M) of the human CD1b protein and the α3 membrane-proximal domain of the human CD1b protein form therebetween a heterodimer that is stabilized by a disulfide bond.

In one aspect, the present invention relates to an antibody-like molecule that specifically binds to first and second targets, wherein said antibody-like molecule comprises:
1) a first antigen-binding fragment that specifically binds to the first target and comprises:
a) the light chain of the first antigen-binding fragment, which light chain comprises a light chain variable domain and a light chain constant domain;
and
b) the heavy chain of the first antigen-binding fragment, which heavy chain comprises a heavy chain variable domain and heavy chain constant domains of antibody that include a first (CH1) heavy chain constant domain and an Fc fragment monomer comprising second (CH2) and third (CH3) heavy chain constant domains;
and
2) a second antigen-binding fragment that specifically binds to the second target and comprises:
(i) a) the light chain of the second antigen-binding fragment, which light chain comprises a light chain variable domain and a constant domain that is β2 microglobulin (β2M) of human CD1b (cluster of differentiation 1) protein with the amino acid sequence of SEQ ID NO: 1;
and
b) the heavy chain of the second antigen-binding fragment, which heavy chain comprises a heavy chain variable domain, a constant domain that is the α3 membrane-proximal domain of the human CD1b protein with the amino acid sequence of SEQ ID NO: 2, and an Fc fragment monomer comprising second (CH2) and third (CH3) heavy chain constant domains;
or
(iI) a) the light chain of the second antigen-binding fragment, which light chain comprises a light chain variable domain and a constant domain that is the α3 membrane-proximal domain of the human CD1b protein with the amino acid sequence of SEQ ID NO: 2;
and
b) the heavy chain of the second antigen-binding fragment, which heavy chain comprises a heavy chain variable domain, a constant domain that is β2 microglobulin (β2M) of the human CD1b protein with the amino acid sequence of SEQ ID NO: 1, and an Fc fragment monomer comprising second (CH2) and third (CH3) heavy chain constant domains;
wherein the β2 microglobulin (β2M) of the human CD1b protein with the amino acid sequence of SEQ ID NO: 1 and the α3 membrane-proximal domain of the human CD1b protein with the amino acid sequence of SEQ ID NO: 2 form therebetween a heterodimer that is stabilized by a disulfide bond.

In the above antibody-like molecule that specifically binds to first and second targets, the variable and constant domains are located in heavy and light chains in the following sequence: the light chain of the first antigen binding fragment:
1) a light chain variable domain,
2) a light chain constant domain;
the heavy chain of the first antigen binding fragment:
1) a heavy chain variable domain,
2) a first (CH1) heavy chain constant domain,
3) a second (CH2) heavy chain constant domain, and
4) a third (CH3) heavy chain constant domain;
the light chain of the second antigen-binding fragment:
1) a light chain variable domain,
2) a first membrane-proximal domain of the human CD1b protein;
the heavy chain of the second antigen-binding fragment:
1) a heavy chain variable domain,
2) a second membrane-proximal domain of the human CD1b protein
3) a second (CH2) heavy chain constant domain, and
4) a third (CH3) heavy chain constant domain.

The antibody-like molecule of the invention refers to a glycoprotein comprising at least two heavy (H) chains and two light (L) chains interconnected by disulfide bonds. The first light chain consists of a light chain variable region (abbreviated referred to in the present description as VL1) and a light chain constant region. Preferably the first light chain is a kappa (κ) light chain, and the constant domain CL is preferably C kappa (κ). The first heavy chain comprises a heavy chain variable region (abbreviated referred to in the present description as VH1) and a heavy chain constant region comprising CH1-CH2-CH3. The second light chain consists of a light chain variable region (abbreviated referred to in the present description as VL2) and a light chain constant region that is represented by the first membrane-proximal domain of the human CD1b protein. The second heavy chain comprises a heavy chain variable region (abbreviated referred to in the present description as VH2) and a heavy chain constant region comprising the second membrane-proximal domain of the human CD1b protein and CH2-CH3.

The antibody-like molecule according to the invention may be an antibody-like molecule of any class (e.g. IgA, IgD, IgE, IgG, and IgM, preferably IgG), or subclass (e.g. IgG1, IgG2, IgG3, IgG4, IgAQ1 and IgA2, preferably IgG1).

VL and VH regions may be further subdivided into hyper-variability regions called complementarity determining regions (CDRs), interspersed between regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of heavy and light chains contain a binding domain that interacts with an antigen.

The constant regions of antibody-like molecule may mediate the binding of immunoglobulin to host tissues or factors, including various cells of the immune system (e.g. effector cells) and the first component (C1q) of the classical complement system.

The term “antigen-binding fragment”, as used in the present description, refers to one or more fragments of antibody-like molecule that retain the ability to specifically bind to an antigen.

“Kabat numbering scheme” or “numbering according to Kabat” as used in the present application refers to the system for numbering of amino acid residues that are more variable (i.e. hypervariable) than other amino acid residues in variable regions of heavy and light chains of antibody-like molecule (Kabat et al. Ann. N.Y. Acad. Sci., 190:382-93 (1971); Kabat et al. Sequences of Proteins of Immunological Interest, Fifth Edition, U.S. Department of Health and Human Services, NIH Publication No. 91-3242 (1991)).

The antibody-like molecule of the present invention “which specifically binds” a target antigen or target refers to an antibody-like molecule that binds an antigen or target with sufficient affinity such that the antibody-like molecule may be used as a diagnostic and/or therapeutic agent targeting a protein or cell or tissue expressing the antigen, and slightly cross-reacts with other proteins.

The term “specifically binds to” a particular polypeptide or an epitope on a particular target polypeptide may be described by example of a molecule having a Kd for the target of at least about 200 nM, or at least about 150 nM, or at least about 100 nM, or at least about 60 nM, or at least about 50 nM, or at least about 40 nM, or at least about 30 nM, or at least about 20 nM, or at least about 10 nM, or at least about 8 nM, or at least about 6 nM, or at least about 4 nM, or at least about 2 nM, or at least about 1 nM, or lower.

In one embodiment, the term “specific binding” refers to binding where a molecule binds to a particular polypeptide or epitope on a particular polypeptide without substantially binding to any other polypeptide or epitope on a polypeptide.

The fragment crystallizable region (“Fe region, Fc”) of an immunoglobulin is the terminal region of an immunoglobulin molecule that interacts with cell surface Fc receptor as well as with some proteins of the complement system. This property allows antibodies or the antibody-like molecule according to the invention to activate the immune system. In IgG, IgA and IgD isotypes, the Fc region is composed of two identical protein fragments from the second and third constant domains of the two heavy chains, respectively; in IgM and IgE isotypes, the Fc contains three heavy chain constant domains (CH2, CH3, and CH4 domains) in each polypeptide chain.

“Fc fragment monomer” is understood to mean an Fc region from the second and third constant domains of either one of the two heavy chains (for IgG, IgA and IgD isotypes); for IgM and IgE isotypes, the Fc monomer contains three constant domains of one of the two heavy chains (CH2, CH3 and CH4 domains).

CD1 (cluster of differentiation 1) refers to a cluster of differentiation 1 molecule that is a component of the immune system located on the surface of various antigen-presenting cells, such as dendritic cells, macrophages and other cells. In the same fashion as MHC classes I, II, CD1 present antigens for recognition by T cells through interaction with the T cell receptor. Unlike MHC classes I, II, CD1 proteins present lipids and derivatives thereof rather than peptides.

The plurality of CD1 variants found in humans are divided into 5 groups: CD1a, CD1b, CD1c, CD1d, CD1e, differing in the structure of the antigen-binding fragment and, consequently, in specificity for lipids of different structures. CD1e proteins, unlike proteins of other groups, are not expressed on the cell surface, but are soluble and are responsible for lipid transport (Kaczmarek, R., Pasciak, M., Szymczak-Kulus, K., & Czerwinski, M. (2017). CD1: A Singed Cat of the Three Antigen Presentation Systems. Archivum Immunologiae et Therapiae Experimentalis, 65(3), 201-214).

CD1 molecules are structurally similar to MHC class I. In a similar fashion, one CD1 molecule is a non-covalent complex consisting of two polypeptide chains: a polymorphic α-chain (sometimes referred to as heavy chain) and a smaller chain called β2 microglobulin (also known as light chain) which is generally not polymorphic.

The α chain forms an antigen-binding region comprising α1 and α2 domains. The α2 domain is followed by the α3 domain located at the C-terminus of the extracellular portion of the α chain of CD1 and forms together with β2 microglobulin a heterodimeric non-covalent complex. Said heterodimeric non-covalent complex composed of the α3 domain of CD1 and β2 microglobulin is referred to in the description of the present invention as a heterodimer of membrane-proximal domains of the CD1b protein.

Membrane-proximal domains of the human CD1b protein are understood to mean the α3 domain of the human CD1b protein and β2 microglobulin of the human CD1b protein.

β2M of the human CD1b protein in the antibody-like molecule according to the invention is β2M of wild-type human CD1b protein with the amino acid sequence of SEQ ID NO: 1.

The α3 membrane-proximal domain of the human CD1b protein in the antibody-like molecule according to the invention is the α3 membrane-proximal domain of the human CD1b protein with the amino acid sequence of SEQ ID NO: 2, which differs from the α3 membrane-proximal domain of wild-type human CD1b protein with the amino acid sequence of SEQ ID NO: 3 in that it has S12K and N59D mutations and GSC elongation at the C-terminus.

In some embodiments of the invention, the antibody-like molecule comprises a CH3 domain of one heavy chain and a CH3 domain of other heavy chain that contact one another via surfaces that are modified to form the antibody-like molecule, wherein the modifications in the CH3 domains of heavy chains are substitutions to provide for heterodimerization.

In some embodiments of the invention, the antibody-like molecule comprises:
a) CH3 domain of one heavy chain, which is modified so that on the surface of the CH3 domain of one heavy chain contacting the surface of the CH3 domain of other heavy chain in the antibody-like molecule, the amino acid residue is substituted with an amino acid residue that has a larger side chain volume, leading to formation of a knob on the surface of the CH3 domain of one heavy chain that can fit into a hole on the surface of the CH3 domain of other heavy chain,
and
b) the CH3 domain of other heavy chain that is modified so that on the surface of the CH3 domain of the second heavy chain contacting the surface ofthe CH3 domain of the first heavy chain in the antibody-like molecule, the amino acid residue is substituted with an amino acid residue that has a smaller side chain volume, leading to formation of a hole on the surface of the CH3 domain of the second heavy chain capable of fitting a knob on the interface of the CH3 domain of the first heavy chain;
wherein said amino acid residue that has a larger side chain volume is selected from a group including arginine (R), phenylalanine (F), tyrosine (Y), tryptophan (W),
and wherein said amino acid residue that has a smaller side chain volume is selected from a group including alanine (A), serine (S), threonine (T), valine (V).

In some embodiments of the invention, the antibody-like molecule comprises a constant domain of the first light chain of antibody, which is selected from CK or CL.

In some embodiments of the invention, the antibody-like molecule comprises CH3 domains of antibody that are modified by introduction of cysteine (C) as an amino acid into the corresponding positions of each CH3 domain so that a disulfide bridge may form between the both CH3 domains.

In some embodiments of the invention, the antibody-like molecule comprises a CH3 domain of one heavy chain, which is modified to form Knob, and a CH3 domain of other heavy chain, which is modified to form Hole, or vice versa.

“Knobs-into-holes” (interactions of the “knobs -into-holes” type) is an approach that enables to circumvent the problem associated with mispaired byproducts. This approach aims at forcing the pairing of two different heavy chains of antibody or antibody-like molecule by way of introducing mutations into the CH3 domains to modify the contact interfaces. On one chain, bulky amino acids were replaced by amino acids with short side chains to create a “hole”. Conversely, amino acids with larger side chains were introduced into the other CH3 domain to create a “knob”. By co-expressing these two heavy chains, a high yield of heterodimer formation (“knob-hole”) versus homodimer formation (“hole-hole” or “knob-knob”) was produced (see, for example, publications of international applications WO9627011 and WO9850431).

In some embodiments of the invention, the antibody-like molecule comprises a CH3 domain of one heavy chain, which has S354C/T366W amino acid substitutions according to the EU numbering scheme for amino acids of antibodies, and a CH3 domain of other heavy chain, which has Y349C/T366S/L368A/Y407V amino acid substitutions according to the EU numbering scheme for amino acids of antibodies.

In some embodiments of the invention, the antibody-like molecule comprises a CH3 domain of one heavy chain, which has Y349C/T366S/L368A/Y407 amino acid substitutions according to the EU numbering scheme for amino acids of antibodies, and a CH3 domain of other heavy chain, which has S354C/T366W amino acid substitutions according to the EU numbering scheme for amino acids of antibodies.

In some embodiments of the invention, the antibody-like molecule comprises an Fc fragment that belongs to IgG.

In some embodiments of the invention, the antibody-like molecule comprises an Fc fragment selected from the group comprising: human IgG1, IgG2, or IgG4.

In some embodiments of the invention, the antibody-like molecule comprises an Fc fragment monomer that comprises substitutions leading to no ADCC, CDC and/or ADCP properties in the antibody-like molecule.

In some embodiments of the invention, the antibody-like molecule comprises an Fc fragment monomer that comprises L234A and L235A substitutions according to the EU numbering scheme for amino acids of antibodies.

In some embodiments of the invention, the antibody-like molecule comprises an Fc fragment monomer that comprises substitutions leading to prolonged action of the antibody-like molecule.

In some embodiments of the invention, the antibody-like molecule comprises an Fc fragment monomer that comprises M252Y, S254T and T256E substitutions according to the EU numbering scheme for amino acids of antibodies.

In some embodiments of the invention, the antibody-like molecule comprises an Fc fragment monomer that comprises substitutions leading to enhanced ADCC, CDC and/or ADCP properties in the antibody-like molecule.

In some embodiments of the invention, the antibody-like molecule comprises an Fc fragment monomer that comprises E345R substitution according to the EU numbering scheme for amino acids of antibodies.

The above mutations in the Fc fragment are numbered according to the EU numbering scheme for amino acids of chains of antibodies (Edelman, G. M., et al., Proc. Natl. Acad. Sci. USA 63 (1969), pp. 78-85; Kabat, E. A., et al., Sequences of Proteins of Immunological Interest, 5th ed., Public Health Service, National Institutes of Health, Bethesda, MD, (1991).

The term “effector function” of antibody or of antibody-like molecule refers to biological activities attributable to the Fc region (native Fc region sequence or to Fc region amino acid variants) of an antibody or of antibody-like molecule, which vary with the isotype of antibody or of antibody-like molecule. Examples of effector functions of antibody or of antibody-like molecule include: Clq binding and complement dependent cytotoxicity; Fc receptor binding; antibody-dependent cell-mediated cytotoxicity (ADCC); phagocytosis; down regulation of cell surface receptors (e.g., B cell receptor, BCR), and B cell activation.

“Antibody-dependent cellular cytotoxicity” or “ADCC” refers to a cell-mediated response, in which nonspecific cytotoxic cells that express Fc receptors (FcR) (for example, natural killer (NK) cells, neutrophils, and macrophages) recognize bound antibody or antibody-like molecule on a target cell and subsequently cause lysis or phagocytosis of the target cell.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMCs), natural killer (NK) cells, monocytes, cytotoxic T cells and neutrophils; with PBMCs and NK cells being preferred.

“Complement dependent cytotoxicity” and “CDC” refer to the ability of a molecule to lyse a target in the presence of complement. The complement activation pathway is initiated by the binding of the first component of the complement system (C1q) to a molecule complexed with a cognate antigen.

In some embodiments of the invention, the antibody-like molecule specifically binds to first and second targets, wherein the target refers to a single antigen.

In some embodiments of the invention, the antibody-like molecule specifically binds to first and second targets, wherein the target is understood to mean two, three or more structurally similar antigens.

One skilled in the art will appreciate that antibodies and antibody-like molecules may have cross-reactivity due to the similarity in protein structures of antigens/targets. For example, antibodies to programmed cell death protein 1 (PD-1) may have cross-reactivity to other members of the CD28 receptor family, including CD28, CTLA-4, ICOS and BTLA as well. In turn, antibodies to GITR (Glucocorticoid-induced TNFR-related protein) may have cross-reactivity to other representatives of the TNFRSF superfamily, including CD137, OX40 and CD27 as well. Cross-reactivity for therapeutic antibodies is typically avoided, for example, the publication of the PCT application WO 2006/121168 provides an antibody that binds to PD-1 but does not substantially bind to CD28, CTLA-4 and human ICOS. However, cross-reactivity is an important feature for a number of therapeutic antibodies; for example, the prior art provides monoclonal antibody ustekinumab that binds to the p40 subunit common to both IL12 and IL23 (Juliane Weber ET AL., Ustekinumab, BioDrugs, 2009, 23(1):53-61. doi: 10.2165/00063030-200923010-00006). Also known are antibodies comprising two antigen-binding fragments, wherein one ofthe antigen-binding fragments binds to 2 antigens IL17A or IL17F, since these interleukins have high percentage of structural identity; thus, these antibodies with two antigen-binding fragments are specific for 3 antigens at once. For example, patent U.S. Ser. No. 10/562,967 provides an antibody that comprises two antigen-binding fragments and is specific for 3 antigens, IL-23p19, IL-17A and IL-17F, whereas the publication of PCT application WO2017188850 provides an antibody that comprises two antigen-binding fragments and is specific for 3 antigens, TNF alfa, IL-17A and IL-17F. Based on the above, the target to which the antibody-like molecule according to the invention binds is understood to mean an antigen (of a protein or other nature) comprising an epitope that is recognized and bound by the antigen-binding site of the antibody-like molecule, or to antigens (of a protein or other nature) comprising epitopes that are recognized and bound by the antigen-binding site of the antibody-like molecule. In case the antibody-like molecule comprises multiple antigen-binding sites that recognize and bind distinct targets (including cases where a single antigen-binding site is able to recognize and bind multiple antigens), then such antibody or antibody-like molecule is considered multispecific (bispecific in the case of 2 antigens, trispecific in the case of 3 antigens, tetraspecific in the case of 4 antigens, and so forth).

The antibody-like molecules according to the invention, which comprise two antigen-binding fragments and specifically bind to at least three antigens, for example, three, four, five, six, seven, eight, nine, etc. antigens, are within the scope of the present invention.

In some embodiments of the invention, the antibody-like molecule specifically binds to first and second targets, wherein the first and second targets may each be independently selected from a group comprising: CD20, BCMA, PD-1, PD-L1, CD47, GD2, AXL, TGF beta, CSF1R, blood clotting factor 9 (FIX), blood clotting factor 10 (FX), TNF alfa, IL17A, IL17F or CD3.

In some embodiments of the invention, the antibody-like molecule is a multispecific antibody-like molecule.

In some embodiments of the invention, the antibody-like molecule is a bispecific, trispecific, tetraspecific, pentaspecific, hexaspecific, heptaspecific, octaspecific or nonaspecific antibody-like molecule.

In some embodiments, the antibody-like molecule is a bivalent bispecific antibody-like molecule.

The application materials provide the following antibody-like molecules: 08-001, 08-002, 08-003, 08-004, 08-005 or 08-006.
08-001 is an antibody-like molecule that specifically binds to PD1 and CD20 and comprises:
1) a first antigen-binding fragment that specifically binds to PD1 and comprises:
a) the light chain of the first antigen-binding fragment, which light chain comprises the light chain variable domain of antibody Prolgolimab (Prolgolimab_VL) with SEQ ID NO: 5 and a light chain constant domain;
and
b) the heavy chain of the first antigen-binding fragment, which heavy chain comprises the heavy chain variable domain of antibody Prolgolimab (Prolgolimab_VH) with SEQ ID NO: 4 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to CD20 and comprises:
a) the light chain of the second antigen-binding fragment, which light chain comprises the light chain variable domain of antibody Ocrelizumab (Ocrelizumab_VL) with SEQ ID NO: 7 and a constant domain that is the α3 membrane-proximal domain of the human CDib protein with the amino acid sequence of SEQ ID NO: 2;
and
b) the heavy chain of the second antigen-binding fragment, which heavy chain comprises the heavy chain variable domain of antibody Ocrelizumab (Ocrelizumab_VH) with SEQ ID NO: 6, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-001 is an antibody-like molecule that specifically binds to PD1 and CD20 and comprises:
1) a first antigen-binding fragment that specifically binds to PD1 and comprises:
a) the light chain of the first antigen-binding fragment with the amino acid sequence of SEQ ID NO: 11 (Prolgolimab_VL_CK);
and
b) the heavy chain of the first antigen-binding fragment with the amino acid sequence of SEQ ID NO: 10 (Prolgolimab_VH_HC_hole);
and
2) a second antigen-binding fragment that specifically binds to CD20 and comprises:
a) the light chain of the second antigen-binding fragment with the amino acid sequence of SEQ ID NO: 13 (Ocrelizumab_VL_CD1b);
and
b) the heavy chain of the second antigen-binding fragment with the amino acid sequence of SEQ ID NO: 12 (Ocrelizumab_VH_b2m_Fc_knob). 08-002 is an antibody-like molecule that specifically binds to CD20 and PD1 and comprises: 1) a first antigen-binding fragment that specifically binds to CD20 and comprises:
a) the light chain of the first antigen-binding fragment, which light chain comprises the light chain variable domain of antibody Ocrelizumab (Ocrelizumab_VL) with SEQ ID NO: 7 and a light chain constant domain;
and
b) the heavy chain of the first antigen-binding fragment, which heavy chain comprises the heavy chain variable domain of antibody Ocrelizumab (Ocrelizumab_VH) with SEQ ID NO: 6 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to PD1 and comprises:
a) the light chain of the second antigen-binding fragment, which light chain comprises the light chain variable domain of antibody Prolgolimab (Prolgolimab_VL) with SEQ ID NO: 5 and a constant domain that is the α3 membrane-proximal domain of the human CD1b protein with the amino acid sequence of SEQ ID NO: 2;
and
b) the heavy chain of the second antigen-binding fragment, which heavy chain comprises the heavy chain variable domain of antibody Prolgolimab (Prolgolimab_VH) with SEQ ID NO: 4, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-002 is an antibody-like molecule that specifically binds to CD20 and PD1 and comprises: 1) a first antigen-binding fragment that specifically binds to CD20 and comprises:
a) the light chain of the first antigen-binding fragment with the amino acid sequence of SEQ ID NO: 15 (Ocrelizumab_VL_CK);
and
b) the heavy chain of the first antigen-binding fragment with the amino acid sequence of SEQ ID NO: 14 (Ocrelizumab_VH_HC_hole);
and
2) a second antigen-binding fragment that specifically binds to PD1 and comprises:
a) the light chain of the second antigen-binding fragment with the amino acid sequence of SEQ ID NO: 17 (Prolgolimab_VL_CD1b);
and
b) the heavy chain of the second antigen-binding fragment with the amino acid sequence of SEQ ID NO: 16 (Prolgolimab_VH_b2m_Fc_knob).
08-003 is an antibody-like molecule that specifically binds to PD1 and CSF1R and comprises:
1) a first antigen-binding fragment that specifically binds to PD1 and comprises:
a) the light chain of the first antigen-binding fragment, which light chain comprises the light chain variable domain of antibody Prolgolimab (Prolgolimab_VL) with SEQ ID NO: 5 and a light chain constant domain;
and
b) the heavy chain of the first antigen-binding fragment, which heavy chain comprises the heavy chain variable domain of antibody Prolgolimab (Prolgolimab_VH) with SEQ ID NO: 4 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to CSF1R and comprises:
a) the light chain of the second antigen-binding fragment, which light chain comprises the light chain variable domain of antibody to CSF1R (Anti-CSF1R_VL) with SEQ ID NO: 9 and a constant domain that is the α3 membrane-proximal domain of the human CD1b protein with the amino acid sequence of SEQ ID NO: 2;
and
b) the heavy chain of the second antigen-binding fragment, which heavy chain comprises the heavy chain variable domain of antibody to CSF1R (Anti-CSF1R_VH) with SEQ ID NO: 8, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-003 is an antibody-like molecule that specifically binds to PD1 and CSF1R and comprises:
1) a first antigen-binding fragment that specifically binds to PD1 and comprises:
a) the light chain of the first antigen-binding fragment with the amino acid sequence of SEQ ID NO: 11 (Prolgolimab_VL_CK);
and
b) the heavy chain of the first antigen-binding fragment with the amino acid sequence of SEQ ID NO: 10 (Prolgolimab_VH_HC_hole);
and
2) a second antigen-binding fragment that specifically binds to CSF1R and comprises:
a) the light chain of the second antigen-binding fragment with the amino acid sequence of SEQ ID NO: 19 (Anti-CSF1R_VL_CD1b);
and
b) the heavy chain of the second antigen-binding fragment with the amino acid sequence of SEQ ID NO: 18 (Anti-CSF1R_VH_b2m_Fc_knob).
08-004 is an antibody-like molecule that specifically binds to CSF1R and PD1 and comprises:
1) a first antigen-binding fragment that specifically binds to CSF1R and comprises:
a) the light chain of the first antigen-binding fragment, which light chain comprises the light chain variable domain of antibody to CSF1R (Anti-CSF1R_VL) with SEQ ID NO: 9 and a light chain constant domain;
and
b) the heavy chain of the first antigen-binding fragment, which heavy chain comprises the heavy chain variable domain of antibody to CSF1R (Anti-CSF1R_VH) with SEQ ID NO: 8 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to PD1 and comprises:
a) the light chain of the second antigen-binding fragment, which light chain comprises the light chain variable domain of antibody Prolgolimab (Prolgolimab_VL) with SEQ ID NO: 5 and a constant domain that is the α3 membrane-proximal domain of the human CD1b protein with the amino acid sequence of SEQ ID NO: 2;
and
b) the heavy chain of the second antigen-binding fragment, which heavy chain comprises the heavy chain variable domain of antibody Prolgolimab (Prolgolimab_VH) with SEQ ID NO: 4, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-004 is an antibody-like molecule that specifically binds to CSF1R and PD1 and comprises:
1) a first antigen-binding fragment that specifically binds to CSF1R and comprises:
a) the light chain of the first antigen-binding fragment with the amino acid sequence of SEQ ID NO: 21 (Anti-CSF1R_VL_CK);
and
b) the heavy chain of the first antigen-binding fragment with the amino acid sequence of SEQ ID NO: 20 (Anti-CSF1R_VH_HC_hole);
and
2) a second antigen-binding fragment that specifically binds to PD1 and comprises:
a) the light chain of the second antigen-binding fragment with the amino acid sequence of SEQ ID NO: 17 (Prolgolimab_VL_CD1b);
and
b) the heavy chain of the second antigen-binding fragment with the amino acid sequence of SEQ ID NO: 16 (Prolgolimab_VH_b2m_Fc_knob).
08-005 is an antibody-like molecule that specifically binds to CD20 and CSF1R and comprises:
1) a first antigen-binding fragment that specifically binds to CD20 and comprises:
a) the light chain of the first antigen-binding fragment, which light chain comprises the light chain variable domain of antibody Ocrelizumab (Ocrelizumab_VL) with SEQ ID NO: 7 and a light chain constant domain;
and
b) the heavy chain of the first antigen-binding fragment, which heavy chain comprises the heavy chain variable domain of antibody Ocrelizumab (Ocrelizumab_VH) with SEQ ID NO: 6 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to CSF1R and comprises:
a) the light chain of the second antigen-binding fragment, which light chain comprises the light chain variable domain of antibody to CSF1R (Anti-CSF1R_VL) with SEQ ID NO: 9 and a constant domain that is the α3 membrane-proximal domain of the human CD1b protein with the amino acid sequence of SEQ ID NO: 2;
and
b) the heavy chain of the second antigen-binding fragment, which heavy chain comprises the heavy chain variable domain of antibody to CSF1R (Anti-CSF1R_VH) with SEQ ID NO: 8, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-005 is an antibody-like molecule that specifically binds to CD20 and CSF1R and comprises: 1) a first antigen-binding fragment that specifically binds to CD20 and comprises:
a) the light chain of the first antigen-binding fragment with the amino acid sequence of SEQ ID NO: 15 (Ocrelizumab_VL_CK);
and
b) the heavy chain of the first antigen-binding fragment with the amino acid sequence of SEQ ID NO: 14 (Ocrelizumab_VH_HC_hole);
and
2) a second antigen-binding fragment that specifically binds to CSF1R and comprises:
a) the light chain of the second antigen-binding fragment with the amino acid sequence of SEQ ID NO: 19 (Anti-CSF1R_VL_CD1b);
and
b) the heavy chain of the second antigen-binding fragment with the amino acid sequence of SEQ ID NO: 18 (Anti-CSF1R_VH_b2m_Fc_knob).
08-006 is an antibody-like molecule that specifically binds to CSF1R and CD20 and comprises:
1) a first antigen-binding fragment that specifically binds to CSF1R and comprises:
a) the light chain of the first antigen-binding fragment, which light chain comprises the light chain variable domain of antibody to CSF1R (Anti-CSF1R_VL) with SEQ ID NO: 9 and a light chain constant domain;
and
b) the heavy chain of the first antigen-binding fragment, which heavy chain comprises the heavy chain variable domain of antibody to CSF1R (Anti-CSF1R_VH) with SEQ ID NO: 8 and antibody heavy chain constant domains that include a first (CH1) heavy chain constant domain and an Fc fragment monomer comprising second (CH2) and third (CH3) heavy chain constant domains;
and
2) a second antigen-binding fragment that specifically binds to CD20 and comprises:
a) the light chain of the second antigen-binding fragment, which light chain comprises the light chain variable domain of antibody Ocrelizumab (Ocrelizumab_VL) with SEQ ID NO: 7 and a constant domain that is the α3 membrane-proximal domain of the human CD1b protein with the amino acid sequence of SEQ ID NO: 2;
and
b) the heavy chain of the second antigen-binding fragment, which heavy chain comprises the heavy chain variable domain of antibody Ocrelizumab (Ocrelizumab_VH) with SEQ ID NO: 6, a constant domain that is β2 microglobulin (β2M) of the human CD1b protein with the amino acid sequence of SEQ ID NO: 1, and an Fc fragment monomer comprising second (CH2) and third (CH3) heavy chain constant domains.
08-006 is an antibody-like molecule that specifically binds to CSF1R and CD20 and comprises:
1) a first antigen-binding fragment that specifically binds to CSF1R and comprises:
a) the light chain of the first antigen-binding fragment with the amino acid sequence of SEQ ID NO: 21 (Anti-CSF1R_VL_CK);
and
b) the heavy chain of the first antigen-binding fragment with the amino acid sequence of SEQ ID NO: 20 (Anti-CSF1R_VH_HC_hole);
and
2) a second antigen-binding fragment that specifically binds to CD20 and comprises:
a) the light chain of the second antigen-binding fragment with the amino acid sequence of SEQ ID NO: 13 (Ocrelizumab_VL_CD1b);
and
b) the heavy chain of the second antigen-binding fragment with the amino acid sequence of SEQ ID NO: 12 (Ocrelizumab_VH_b2m_Fc_knob).

These antibody-like molecules are given for illustrative purposes to confirm the operability of the format of the antibody-like molecule according to the invention, as well as surprising properties thereof.

Such antibody-like molecules should not be construed as somehow limiting the antibody-like molecule of the invention.

The yield parameters of the product with the correct heterodimeric assembly of two distinct heavy chains and the correct pairing between two distinct light chains and cognate heavy chains do not depend on the heavy and light chain variable fragments of the antibody-like molecule and specificity thereof for antigens.

The antibody-like molecules according to the invention may be used to treat various diseases, in particular, oncological diseases, autoimmune diseases or diseases that are associated with blood clotting (coagulation) disorder.

### Nucleic Acid Molecule

In one aspect, the present invention relates to a nucleic acid that encodes the above antibody-like molecule.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

Unless otherwise indicated, the term nucleotide sequence encompasses its complement. Thus, a nucleic acid having a particular sequence should be understood as one which encompasses the complementary strand thereof with the complementary sequence thereof.

In any one of said embodiments, the nucleic acid molecules may be isolated.

An “isolated” nucleic acid molecule is one which is identified and separated from at least one nucleic acid molecule-impurity. An isolated nucleic acid molecule is different from the form or set in which it is found under natural conditions. Thus, an isolated nucleic acid molecule is different from a nucleic acid molecule that exists in cells under natural conditions.

In one aspect, the present invention relates to a nucleic acid molecule comprising a nucleotide sequence that encodes an amino acid sequence selected from SEQ ID NO: 22-33. A nucleic acid molecule may also comprise any combination of said nucleotide sequences.

In some embodiments of the invention, the isolated nucleic acid is DNA.

In one embodiment, the present invention relates to a nucleic acid molecule that encodes the amino acid sequence of the light or heavy chain of the above antibody-like molecule according to the invention, selected from:
a nucleic acid that encodes the amino acid sequence of the light chain of the first antigen-binding fragment, which comprises a light chain variable domain and a light chain constant domain;
a nucleic acid that encodes the amino acid sequence of the heavy chain of the first antigen-binding fragment, which heavy chain comprises a heavy chain variable domain and heavy chain constant domains of the antibody that include a first (CH1) heavy chain constant domain and an Fc fragment monomer comprising second (CH2) and third (CH3) heavy chain constant domains;
a nucleic acid that encodes the amino acid sequence of the light chain of the second antigen-binding fragment, which light chain comprises a light chain variable domain and a constant domain that is β2 microglobulin (β2M) of human CD1b (cluster of differentiation 1) protein with the amino acid sequence of SEQ ID NO: 1;
a nucleic acid that encodes the amino acid sequence of the heavy chain of the second antigen-binding fragment, which heavy chain comprises a heavy chain variable domain, a constant domain that is the α3 membrane-proximal domain of the human CD1b protein with the amino acid sequence of SEQ ID NO: 2, and an Fc fragment monomer comprising second (CH2) and third (CH3) heavy chain constant domains;
a nucleic acid that encodes the amino acid sequence of the light chain of the second antigen-binding fragment, which light chain comprises a light chain variable domain and a constant domain that is the α3 membrane-proximal domain of the human CD1b protein with the amino acid sequence of SEQ ID NO: 2;
or
a nucleic acid that encodes the amino acid sequence of the heavy chain of the second antigen-binding fragment, which heavy chain comprises a heavy chain variable domain, a constant domain that is β2 microglobulin (β2M) of the human CD1b protein with the amino acid sequence of SEQ ID NO: 1, and an Fc fragment monomer comprising second (CH2) and third (CH3) heavy chain constant domains.

The nucleic acid molecule may also comprise a combination of the above nucleotide sequences necessary to produce the antibody-like molecule according to the invention.

As would be appreciated by those skilled in the art, because of the redundancy of the genetic code, a variety of different DNA sequences can encode the amino acid sequences of the light chain or heavy chain of the antibody-like molecule according to the invention or fragments thereof (VH, VL, CDR, etc.). It is well within the skill of those trained in the art to create these alternative DNA sequences encoding one and the same amino acid sequences. Such variant DNA sequences are within the scope of the present invention.

The nucleic acid molecule according to the present invention may be isolated from any source that produces the antibody-like molecule according to the invention. In certain embodiments of the invention, the nucleic acid molecule of the invention may be synthesized by way of chemical synthesis, rather than isolated.

In some embodiments of the invention, the nucleic acid is a nucleic acid that encodes the amino acid sequence of the heavy chain of the first antigen-binding fragment of antibody-like molecules 08-001 and 08-003 (Prolgolimab_VH_HC_hole) and comprises a nucleotide sequence with SEQ ID NO: 22.

In some embodiments of the invention, the nucleic acid is a nucleic acid that encodes the amino acid sequence of the light chain of the first antigen-binding fragment of antibody-like molecules 08-001 and 08-003 (Prolgolimab_VL_CK) and comprises a nucleotide sequence with SEQ ID NO: 23.

In some embodiments of the invention, the nucleic acid is a nucleic acid that encodes the amino acid sequence of the heavy chain of the second antigen-binding fragment of the antibody-like molecules 08-001 and 08-006 (Ocrelizumab_VH_b2m_Fc_knob) and comprises a nucleotide sequence with SEQ ID NO: 24.

In some embodiments of the invention, the nucleic acid is a nucleic acid that encodes the amino acid sequence of the light chain of the second antigen-binding fragment of antibody-like molecules 08-001 and 08-006 (Ocrelizumab_VL_CD1b) and comprises a nucleotide sequence with SEQ ID NO: 25.

In some embodiments of the invention, the nucleic acid is a nucleic acid that encodes the amino acid sequence of the heavy chain of the first antigen-binding fragment of antibody-like molecules 08-002 and 08-005 (Ocrelizumab_VH_HC_hole) and comprises a nucleotide sequence with SEQ ID NO: 26.

In some embodiments of the invention, the nucleic acid is a nucleic acid that encodes the amino acid sequence of the light chain of the first antigen-binding fragment of antibody-like molecules 08-002 and 08-005 (Ocrelizumab_VL_CK) and comprises a nucleotide sequence with SEQ ID NO: 27.

In some embodiments of the invention, the nucleic acid is a nucleic acid that encodes the amino acid sequence of the heavy chain of the second antigen-binding fragment of antibody-like molecules 08-002 and 08-004 (Prolgolimab_VH_b2m_Fc_knob) and comprises a nucleotide sequence with SEQ ID NO: 28.

In some embodiments of the invention, the nucleic acid is a nucleic acid that encodes the amino acid sequence of the light chain of the second antigen-binding fragment of antibody-like molecules 08-002 and 08-004 (Prolgolimab_VL_CD1b) and comprises a nucleotide sequence with SEQ ID NO: 29.

In some embodiments of the invention, the nucleic acid is a nucleic acid that encodes the amino acid sequence of the heavy chain of the second antigen-binding fragment of antibody-like molecules 08-003 and 08-005 (Anti-CSF1R_VH_b2m_Fc_knob) and comprises a nucleotide sequence with SEQ ID NO: 30.

In some embodiments of the invention, the nucleic acid is a nucleic acid that encodes the amino acid sequence of the light chain of the second antigen-binding fragment of antibody-like molecules 08-003 and 08-005 (Anti-CSFIR_VL_CD1b) and comprises a nucleotide sequence with SEQ ID NO: 31.

In some embodiments of the invention, the nucleic acid is a nucleic acid that encodes the amino acid sequence of the heavy chain of the first antigen-binding fragment of antibody-like molecules 08-004 and 08-006 (Anti-CSF1R_VH_HC_hole) and comprises a nucleotide sequence with SEQ ID NO: 32.

In some embodiments of the invention, the nucleic acid is a nucleic acid that encodes the amino acid sequence of the light chain of the first antigen-binding fragment of antibody-like molecules 08-004 and 08-006 (Anti-CSF1R_VL_CK) and comprises a nucleotide sequence with SEQ ID NO: 33.

The nucleic acid molecules may be used to express the antibody-like molecule according to the invention.

### Vector

In one aspect, the present invention relates to an expression vector comprising any one of the above nucleic acid molecules that encode the corresponding amino acid sequences of the antibody-like molecule according to the invention. The present invention relates to a vector suitable for the expression of any one of nucleotide sequences described herein.

The term “vector” as used herein means a nucleic acid molecule capable of transporting another nucleic acid to which it has been linked.

As used in the present description, the term “expression” is defined as the transcription and/or translation of a particular nucleotide sequence driven by its promoter.

The present invention relates to vectors comprising nucleic acid molecules that encode any one of the above antibody-like molecules or structural portions thereof selected from:
a nucleic acid that encodes the amino acid sequence of the light chain of the first antigen-binding fragment, which comprises a light chain variable domain and a light chain constant domain;
a nucleic acid that encodes the amino acid sequence of the heavy chain of the first antigen-binding fragment, which heavy chain comprises a heavy chain variable domain and heavy chain constant domains of the antibody that include a first (CH1) heavy chain constant domain and an Fc fragment monomer comprising second (CH2) and third (CH3) heavy chain constant domains;
a nucleic acid that encodes the amino acid sequence of the light chain of the second antigen-binding fragment, which light chain comprises a light chain variable domain and a constant domain that is β2 microglobulin (β2M) of human CD1b (cluster of differentiation 1) protein with the amino acid sequence of SEQ ID NO: 1;
a nucleic acid that encodes the amino acid sequence of the heavy chain of the second antigen-binding fragment, which heavy chain comprises a heavy chain variable domain, a constant domain that is the α3 membrane-proximal domain of the human CD1b protein with the amino acid sequence of SEQ ID NO: 2, and an Fc fragment monomer comprising second (CH2) and third (CH3) heavy chain constant domains;
a nucleic acid that encodes the amino acid sequence of the light chain of the second antigen-binding fragment, which light chain comprises a light chain variable domain and a constant domain that is the α3 membrane-proximal domain of the human CD1b protein with the amino acid sequence of SEQ ID NO: 2;

or
a nucleic acid that encodes the amino acid sequence of the heavy chain of the second antigen-binding fragment, which heavy chain comprises a heavy chain variable domain, a constant domain that is β2 microglobulin (β2M) of the human CD1b protein with the amino acid sequence of SEQ ID NO: 1, and an Fc fragment monomer comprising second (CH2) and third (CH3) heavy chain constant domains.

In some embodiments of the invention, the vector is a plasmid, i.e. a circular double stranded piece of DNA into which additional DNA segments may be inserted.

In some embodiments of the invention, the vector is a viral (expression) vector, wherein additional DNA segments may be inserted into the viral genome.

In some embodiments of the invention, the vectors are capable of autonomous replication in a host cell into which they are introduced (e.g. bacterial vectors having a bacterial site of replication origin and episomal vectors). In further embodiments of the invention, vectors (e.g. non-episomal vectors) may be integrated into the genome of a host cell upon introduction into a host cell, and thereby are replicated along with the host gene. Moreover, certain vectors are capable of directing the expression of genes to which they are operably linked. Such vectors are referred to herein as “recombinant expression vectors” (or simply, “expression vectors”).

In some embodiments of the invention, expression vectors include plasmids, retroviruses, adenoviruses, adeno-associated viruses (AAVs), plant viruses, such as cauliflower mosaic virus, tobacco mosaic virus, cosmids, YACs, and the like. DNA molecules may be inserted into a vector such that transcriptional and translational control sequences within the vector serve their intended function of regulating the transcription and translation of DNA. An expression vector and expression control sequences may be chosen to be compatible with the expression host cell used.

In one embodiment of the invention, DNA molecules encoding partially or fully heavy and light chain sequences can be inserted into distinct vectors.

In one embodiment, any combination of the above DNA molecules is introduced into the same expression vector.

In one embodiment of the invention, DNA molecules may be introduced into an expression vector by standard methods (e.g. ligation of complementary restriction sites on a gene fragment of antibody or antibody-like molecule and vector, or blunt end ligation if no restriction sites are present).

In some embodiments of the invention, a suitable vector is one that includes restriction sites such that any VH or VL sequence can easily be inserted and expressed, as described above. A recombinant expression vector may also encode a signal peptide that facilitates secretion of the chain of antibody-like molecule from a host cell. The gene of an antibody-like molecule chain may be cloned into a vector such that the signal peptide is linked in-frame to the amino terminus of an immunoglobulin chain. A signal peptide may be an immunoglobulin signal peptide or a heterologous signal peptide (i.e. a signal peptide from a non-immunoglobulin protein).

In some embodiments of the invention, the vector may include an expression control sequence. The term “expression control sequence” as used in the present description refers to polynucleotide sequences that are necessary to effect the expression and processing of coding sequences to which they are inserted.

It will be understood by those skilled in the art that the design of an expression vector, including the selection of expression control sequences, may depend on such factors as the choice of the type of a host cell to be transformed, the required level of expression of antibody or antibody-like molecule, and so forth. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such expression control sequences differs depending upon the host organism; in prokaryotes, such expression control sequences typically include a promoter, a ribosome binding site, as well as transcription termination sequences; in eukaryotes, such expression control sequences typically include promoters and transcription termination sequences. Preferred expression control sequences for an expression host cell in a mammal include viral elements that ensure high levels of protein expression in mammalian cells, such as promoters and/or enhancers derived from a retroviral LTR, cytomegalovirus (CMV) (such as a CMV promoter/enhancer), simian virus 40 (SV40) (such as a SV40 promoter/enhancer), adenovirus, (e.g. the major late promoter adenovirus (AdMLP)), polyomavirus and strong mammalian promoters such as TTR promoter, native immunoglobulin promoter or actin promoter. Expression control sequences encompass at least all components whose presence is important for expression and processing.

In some embodiments of the invention, in addition to antibody-like molecule chain genes and expression control sequences, the recombinant expression vectors of the invention may carry additional sequences, such as sequences that regulate replication of a vector in host cells (e.g. origins of replication) and selectable marker genes. The selectable marker gene facilitates the selection of host cells into which a vector has been introduced.

### Host Cell

In one aspect, the present invention relates to a method for producing a host cell to produce any one of the above antibody-like molecules according to the invention, and comprises transforming the cell with the above vector or vectors that comprise a combination of the above nucleotide sequences necessary to produce the above antibody-like molecule according to the invention.

In one aspect, the present invention relates to a host cell to produce any one of the above antibody-like molecules according to the invention that comprises a combination of the above nucleotide sequences necessary to produce the above antibody-like molecule according to the invention.

The term “host cell” as used herein refers to a cell into which a recombinant expression vector has been introduced. The present invention relates to host cells, which may include, for example, the above-described vector according to the invention. The present invention further relates to host cells that comprise, for example, a nucleotide sequence encoding a heavy chain, a nucleotide sequence encoding a light chain, or both. It should be understood that “host cell” refers not only to a particular subject cell but to the progeny of such cell as well. Since modifications may occur in succeeding generations due to either mutation or environmental influences, such progeny may not, in fact, be identical to a parental cell; however, such cells are still included within the scope of the term “host cell” as used herein.

Nucleic acid molecules encoding the above antibody-like molecule according to the invention and vectors comprising these nucleic acid molecules can be used for transfection of a mammalian cell, plant cell, bacterial cell or yeast cell. Transfection may be carried out by any known method for introducing polynucleotides into a host cell. Methods for introducing heterologous polynucleotides into mammalian cells are well known in the art and include dextran-mediated transfection, cationic polymer-nucleic acid complex transfection, calcium phosphate precipitation, polybrene-mediated transfection, protoplast fusion, encapsulation of the polynucleotides in liposomes, and direct microinjection of DNA into nuclei. In addition, the nucleic acid molecules may be introduced into mammalian cells by viral (expression) vectors.

Mammalian cell lines used as hosts for transformation are well known in the art and include a plurality of immortalized cell lines available. These include, e.g., Chinese hamster ovary (CHO) cells, NSO cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549, SK-HEP1, HUH7, Hep-RG cells and a number of other cell lines. Cell lines are selected by way of determining which cell lines have high expression levels and provide for necessary characteristics of the protein being produced. Other cell lines that may be used are insect cell lines, such as Sf9 or Sf21 cells. When the recombinant expression vectors encoding the above antibody-like molecule of the invention are introduced into mammalian host cells, the antibody-like molecule is produced by way of culturing the host cells for a period of time sufficient to express the antibody-like molecule of the invention in the host cells, or, more preferably, secrete the antibody-like molecule into the culture medium in which the host cells are being cultured. The above antibody-like molecule of the invention may be isolated from culture medium using standard protein purification techniques. Plant host cells include e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

Furthermore, level of production of the above antibody-like molecule of the invention from a producing cell line may be enhanced using a number of known techniques. For example, the glutamine synthetase gene expression system (the GS system) is a common approach for enhancing expression under certain conditions.

It is likely that the above antibody-like molecule of the invention in different cell lines will have different glycosylation patterns from one another. However, the above antibody-like molecule of the invention encoded by nucleic acid molecules described herein, or comprising amino acid sequences provided herein are part of the present invention, regardless of the glycosylation of the binding molecules, and, in general, regardless of the presence or absence of post-translational modifications.

The above host cell does not relate to a host cell produced using human embryos.

The above host cell does not relate to a host cell produced by modifying the genetic integrity of human germline cells.

### Method for Producing Antibody-Like Molecules

In one aspect, the present invention relates to a method for producing any one of the above antibody-like molecules, wherein the method includes the steps of:
a) transforming a host cell
   with expression vectors comprising nucleic acid molecules encoding the light and heavy chains of the first antigen-binding fragment of the antibody-like molecule,
   with expression vectors comprising nucleic acid molecules encoding the light and heavy chains of the second antigen-binding fragment of the antibody-like molecule,
b) culturing the host cell under conditions suitable for synthesis of said antibody-like molecule; and
c) isolating said antibody-like molecule from cell culture.

### BRIEF DESCRIPTION OF DRAWINGS

FIG. 1 is an electrophoregram of samples of antibody-like molecules following purification from culture liquid. 7.5% PAAG, non-reducing conditions.

M—standard marker of Precision Plus Protein™ Dual Color Standards (BIO-RAD), the molecular weights of the corresponding lanes are indicated as kDa in the column to the left of the gel;
1—08-001;
2—08-002;
3—08-003;
4—08-004;
5—08-005;
6—08-006.

FIG. 2 is an electrophoregram of samples of antibody-like molecules following purification from culture liquid. 12% PAAG, reducing conditions.

M—standard marker of Precision Plus Protein™ Dual Color Standards (BIO-RAD), the molecular weights of the corresponding lanes are indicated as kDa in the column to the left of the gel;
1—08-001;
2—08-002;
3—08-003;
4—08-004;
5—08-005;
6—08-006.

FIG. 3 is an electrophoregram of samples of antibody-like molecules before and following cleavage by GingisKHAN protease. 7.5% PAAG, non-reducing conditions.

M—standard marker of Precision Plus Protein™ Dual Color Standards (BIO-RAD), the molecular weights of the corresponding lanes are indicated as kDa in the column to the left of the gel;
1—08-001 intact sample;
2—08-001 following proteolysis;
3—08-002 intact sample;
4—08-002 following proteolysis;
5—08-003 intact sample;
6—08-003 following proteolysis.

FIG. 4 is an electrophoregram of samples of antibody-like molecules before and following cleavage by GingisKHAN protease. 7.5% PAAG, non-reducing conditions.

M—standard marker of Precision Plus Protein™ Dual Color Standards (BIO-RAD), the molecular weights of the corresponding lanes are indicated as kDa in the column to the left of the gel;
1—08-004 intact sample;
2—08-004 following proteolysis;
3—08-005 intact sample;
4—08-005 following proteolysis;
5—08-006 intact sample;
6—08-006 following proteolysis.

FIG. 5 is an electrophoregram of samples of bispecific and monospecific antibody-like molecules following cleavage by GingisKHAN protease. 7.5% PAAG, non-reducing conditions.

M—standard marker of Precision Plus Protein™ Dual Color Standards (BIO-RAD), the molecular weights of the corresponding lanes are indicated as kDa in the column to the left of the gel;
1—bispecific antibody-like molecule comprising variable domains of antibody Prolgolimab and of antibody Ocrelizumab and a dimerization unit of membrane-proximal domains of the CD1b protein;
2—bispecific antibody-like molecule comprising variable domains of antibody Prolgolimab and of anti-CSF1R and a dimerization unit of membrane-proximal domains of the CD1b protein;
3—antibody Prolgolimab;
4—monospecific antibody-like molecule comprising a dimerization unit based on membrane-proximal domains of the CD1b protein and variable fragments of antibody Prolgolimab;
5—monospecific antibody comprising variable domains of antibody Ocrelizumab.

### EXAMPLES

The following examples are provided for better understanding of the invention. These examples are for purposes of illustration only and are not to be construed as limiting the scope of the invention in any manner.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Materials and General Methods

### Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 1989.

The molecular biological reagents were used according to the manufacturer protocols.

### Gene Synthesis

Desired gene segments were prepared from oligonucleotides made by chemical synthesis. The gene segments of 300-4000 bp long, which were flanked by singular restriction sites, were assembled by annealing and ligation of oligonucleotides including PCR amplification and subsequently cloned via the specified restriction sites. The DNA sequences of the subcloned gene fragments were confirmed by DNA sequencing.

### DNA Sequence Determination

DNA sequences were determined by Sanger sequencing.

### DNA and Protein Sequence Analysis and Sequence Data Management

Ylab2 (Biocad) software package was used for sequence creation, mapping, analysis, annotation and illustration.

### Expression Vectors

Expression plasmid variants were applied for transient expression of the subject antibodies, antibody-like proteins and antigens in eukaryotic cells (e.g. CHO cells). Beside the expression cassette for a target protein, the (plasmids) vectors contained: an origin of replication which allows replication of said plasmid in E. coli, genes which confer resistance in E. coli to various antibiotics (e.g., to ampicillin and kanamycin).

The fusion genes comprising the described chains of antibody or antibody-like molecules as described above were generated by PCR and/or gene synthesis and assembled by known methods and techniques by way of connection of the according nucleic acid segments, e.g. using unique restriction sites in the corresponding vectors. The subcloned nucleic acid sequences were verified by DNA sequencing. The necessary amounts of plasmids for transient transfection were generated in E. coli cell cultures and isolated using known techniques.

### Generation and Purification of Recombinant Antigens in Suspension Culture of Mammalian Cells

Recombinant proteins were produced in established cell line cells obtained from Chinese hamster ovary cells (CHO line). Suspension culture was conducted in flasks on orbital incubator shaker using serum-free media supplemented with 8 mM L-glutamine and 1 g/l pluronic 68. For transient expression, cells at a concentration of 2-2.2×10⁶ cells/ml were transfected using linear polyethyleneimine. 9 days following transfection, culture liquid was separated from cells by filtration through a 0.22 μm filter.

Histidine-tagged proteins were purified by metal chelate chromatography. The purified proteins were filtered through 0.22 m and stored at −70° C.

The purity of the resulting protein solution was evaluated using SDS gel electrophoresis, electrophoresis was performed in denaturing 12% PAAG in the presence of mercaptoethanol and in denaturing 7.5% PAAG in the absence of mercaptoethanol.

### Production of Antibodies and Antibody-Like Proteins in Suspension Culture of Mammalian Cells

Control antibodies, antibody-like molecules according to the invention were produced in established cell line cells obtained from Chinese hamster ovary cells (CHO line). Suspension culture was conducted in flasks on orbital incubator shaker using serum-free media supplemented with 8 mM L-glutamine and 1 g/l pluronic 68. For transient expression, cells at a concentration of 2-2.2×10⁶ cells/ml were transfected using linear polyethyleneimine (hereinafter referred to as PEI). DNA/PEI ratio was 1:3/1:10. 9 days following transfection, the culture fluid was separated from the cells by filtration through a 0.5/0.22 μm deep-bed filter, and the protein titer was then measured on ForteBio using the standard methodology. The clarified culture liquid was passed through a Protein A affinity sorbent column at 10-20 mg per ml of the sorbent, the column was equilibrated with phosphate-buffered saline (PBS, pH 7.4). The column was then washed with 5 column volumes of PBS to remove non-specifically binding components. Bound protein was eluted using 0.1 M glycine buffer (pH 3). The principal protein elution peak was collected and adjusted to pH 6.0 with 1 M Tris buffer (pH 8). All stages were conducted under 110 cm/h flow rate. Protein was then dialyzed into acetate buffer (pH 5.0), filtered (0.22 μm), transferred into tubes and stored at −70° C.

The purity of the resulting protein solution was evaluated using SDS gel electrophoresis under reducing and non-reducing conditions, as well as using size-exclusion high-performance liquid chromatography (SE HPLC).

SE HPLC was performed on a TSK-Gel G3000SWXL column, 7.8×300 mm, particle size: 5 μm, pore size: 250A, and a TSK-Gel Guard SWxl pre-column.

### Example 1. Selection of CH1/CK Domain Substitution in Antibody-Like Molecule

To ensure proper heterodimerization of heavy and light chains in the antibody-like molecule, one of the two pairs of CHUCK domains was substituted with structurally identical domains from other proteins.

This resulted in a dimerization unit consisting of: 1) Natural sequence of β2 microglobulin (β2M) of the human CD1b protein with the amino acid sequence of SEQ ID NO: 1 and 2) α3 membrane-proximal domain of human CD1b with S12K and N59D mutations and GSC elongation at the C-terminus, which has the amino acid sequence of SEQ ID NO: 2. This dimerization unit within the antibody-like molecule allowed for producing functional antibody-like molecules of sufficient purity with distinct variable domains (shown in the examples below), and also increased the temperature stability of the molecules and the yield of a correctly assembled antibody-like molecule.

### Example 2. Production of Genetic Constructs for Generation of Antibody-Like Molecules

To produce constructs encoding sequences of light and heavy chains of the first antigen-binding fragment of the antibody-like molecule specifically binding to the first target, PCR products comprising the genes of variable domains of heavy and light chains of the antibody-like molecule were generated using primers comprising restriction sites. The heavy chain variable domain was cloned into the vector pSXn-HChole-NR_VH1 using SalI/XbaI restriction sites. The light chain variable domain was cloned into the vector pSXn-CL-BR_VL1 using Sal/XbaI restriction sites.

To produce structures encoding sequences of light and heavy chains of the second antigen-binding fragment of the antibody-like molecule specifically binding to the second target, synthesized were constructs comprising sequences encoding variable domains of heavy and light chains of the antibody-like molecule, fused to membrane-proximal domains of the human CD1b protein (https://www.rcsb.org/structure/5wll) including modifications.

The sequence was synthesized from oligonucleotides by PCR using primers comprising restriction sites. The heavy chain variable domain with the first membrane-proximal domain of the human CD1b sequence including modifications was cloned into the vector pSX-FCknob-PR using SalI/XbaI restriction sites. The light chain variable domain with the second membrane-proximal domain of the human CD1b sequence including or free of modifications was cloned into the vector pSX-HR using SalI/XbaI restriction sites.

The above four vectors were combined at the transfection step to produce the antibody-like molecules according to the invention.

The required quantities of all of the above plasmids were generated in E. Coli cells and purified using Maxiprep Qiagen kit.

### Example 3. Generation of Antibody-Like Molecules Comprising Dimerization Unit of Human CD1b Protein

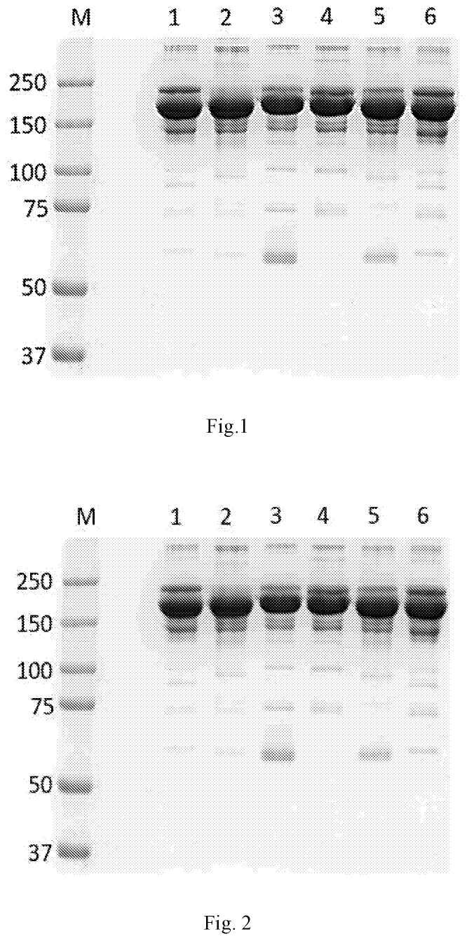

To check for the universality of the subject approach as a platform solution for assembling antibody-like molecules using any pair of antigen-binding fragments (hereinafter light and heavy chain variable fragments), we chose three random pairs of light and heavy chain variable fragments from known antibodies (see Table 1) and used them to generate 6 antibody-like molecules. Table 1 shows the results of productivity for the produced proteins. FIGS. 1 and 2 show the results of SDS gel electrophoresis of the generated samples under non-reducing and reducing conditions. For all six samples in non-reducing conditions there is present a major band at about 150 kDa, corresponding to a full-length molecule. Table 1 also shows the results of the sample purity according to SE HPLC.

TABLE 1
Productivity and purity of antibody-like molecules produced
using dimerization unit of modified α3 domain of the human
CD1b protein and β2-microglobulin of the human CD1b protein.

| | | Second pair of | | | | |
|---|---|---|---|---|---|---|
| | First pair of | light and heavy | | | | |
| | light and | chain variable | | | | |
| | heavy chain | fragments and | | | | |
| | variable | dimerization | | Purity by SE HPLC | | |
| | fragments | unit of human | Productivity, | Aggregates, | Monomer, | Fragments, |
| Sample | within Fab | CD1b protein | day 9, mg/l | % | % | % |
| 08-001 | Prolgolimab | Ocrelizumab | 218.2 | 5.4 | 93.6 | 1.0 |
| 08-002 | Ocrelizumab | Prolgolimab | 198.6 | 6.3 | 92.9 | 0.8 |
| 08-003 | Prolgolimab | Anti-CSF1R | 205.9 | 6.3 | 90.5 | 3.2 |
| 08-004 | Anti-CSF1R | Prolgolimab | 220.6 | 6.0 | 93.0 | 1.1 |
| 08-005 | Ocrelizumab | Anti-CSF1R | 161.7 | 6.8 | 91.5 | 1.6 |
| 08-006 | Anti-CSF1R | Ocrelizumab | 147.8 | 7.6 | 90.7 | 1.7 |

Accordingly, the antibody-like molecules with a dimerization unit according to the invention having various combinations of antigen-binding fragments show high purity and productivity.

### Example 4. Determination of Affinity of Full-Length Antibody-Like Molecules on Forte Bio Octet RED 384

To confirm that the generated antibody-like molecules have not lost the antigen-binding ability thereof, we conducted an affinity analysis on Forte Bio Octet RED 384. For antibody-like molecules 08-001 and 08-002, we measured affinity to the extracellular domain of the human PD-1 protein (hPDlex-H6F) and to the biotinylated peptide [NH2]CEPANPSEKNSPSTQYCYSIQS[CH2CH2]biotin comprising in the amino acid sequence a fragment of the human CD20 sequence (hereinafter referred to as CD20 peptide); for antibody-like molecules 08-003 and 08-004, we measured affinity to the extracellular domain of the human PD-1 protein (hPD1ex-H6F) and to the extracellular domain of the human CSF1R protein (hCSF1R_His); for antibody-like molecules 08-005 and 08-006, we measured affinity to the extracellular domain of the human CSF1Rprotein (hCSF1R_His) and to CD20 peptide.

As the hCSF1R antigen, we used a sequence of amino acids 20-512 (hereinafter referred to as AAs) of human CSF1R protein (Macrophage colony-stimulating factor 1 receptor (Homo sapiens), UNIPROT ID P07333) with C-terminal His-tag and FLAG-tag, molecular weight−57.4 kDa. As the hPD-lex-H6F antigen, we used a sequence of AAs 21-170 of human PD-1 protein (Programmed cell death protein 1 (Homo sapiens), UNIPROT ID Q15116) with C-terminal His-tag and FLAG-tag, molecular weight−20.6 kDa.

Genetic sequences encoding antigens were synthesized de novo, cloned into an expression vector, generated in CHO cells and purified using affinity chromatography as described in general examples.

Experiments were carried out using a kinetic buffer solution (hereinafter referred to as 1×KB); volume fraction of added Tween 20 was 0.1%; mass fraction of added BSA (bovine serum albumin) was 0.1%; pH 7.4. Before the measurement, the ProA sensors were regenerated with a solution of 50 mM glycine and hydrochloric acid, pH 1.8 (5 seconds in the regenerating solution, 5 seconds in 1×KB, three repetitions).

In the case of the hPDlex-H6F and hCSF1R_His antigens, the Protein A (ProA) biosensors (ForteBio) were immersed in a solution with antibody-like molecules at a concentration of 10 g/ml for 300 seconds to immobilize same. The baseline was recorded in 1×KB for 120 seconds. Sensors loaded with the antibody-like molecule were then immersed into wells containing a solution of the target antigen (analyte) in a kinetic buffer for 300 seconds. Measurements were performed for solutions with hPDlex-H6F analyte at concentrations of 10 g/ml (485.4 nM), for solutions with hCSF1R_His analyte at concentrations of 10 g/ml (174.2 nM). Complex dissociation in 1×KB was then detected for 600 seconds.

The reference sensors went through all the steps as the sensors used to record analyte sensograms did, with the exception of the association step—at the association step, the sensors were immersed in 1×KB solution without analyte (the reference sensor signals were measured in parallel with the recording of the main sensograms). The reference signal was subtracted from the signal received on sensors interacting with the analyte during the processing of sensograms.

To check the nonspecific interaction between the analyte and the sensors, we used a sensor non-loaded with antibody-like molecule (at the loading step, the sensor was immersed in 1×KB solution; all other steps are identical to those used for the sensor loaded with antibody-like molecule).

In the case of biotinylated CD2 peptide, the peptide at a concentration of 2.5 μg/ml was immobilized on the surface of SAX sensors (High Precision Streptavidin (SAX) biosensors, ForteBio) for 300 seconds. The baseline was recorded in a 1×KB solution for 120 seconds. Sensors loaded with peptide were then immersed into wells containing the solution of antibody-like molecule (analyte) in 1×KB for 300 seconds. Measurements were performed for solutions with analyte (antibody-like molecule in case of CD20 peptide) at concentrations of300 g/ml and 75 g/ml. Complex dissociation was recorded in 1×KB for 60 seconds.

All measurements were carried out at 30° C., orbital mixing speed was 1000 revolutions per minute.

To obtain numerical values of kinetic constants (kon is the on/association rate constant, kdis is the dissociation rate constant, KD is the equilibrium dissociation constant or affinity constant), the resulting sensograms were processed according to the 1:1 interaction model using Global Fit (selection of one set of kon, kdis, KD constants to analyze several sensograms of different concentrations) of the ForteBio Octet Data Analysis 9.0 software. The results are shown in Table 2.

TABLE 2
Kinetic constants kon, kdis, KD for interaction between antibody-
like molecules 08-001-08-006 and target antigens.

| Antibody- | | | | | |
|---|---|---|---|---|---|
| like | | KD | kon | kdis | |
| molecule | Antigen | (M) | (M−1c−1) | (c−1) | R\hat{ }2 |
| 08-001 | hPD1ex-H6F | 1.59E−08 | 3.78E+04 | 6.01E−04 | 0.9214 |
| | CD20 peptide | 8.48E−07 | 1.64E+05 | 1.39E−01 | 0.9573 |
| 08-002 | hPD1ex-H6F | 1.69E−08 | 3.90E+04 | 6.58E−04 | 0.9223 |
| | CD20 peptide | 8.45E−07 | 1.17E+05 | 9.86E−02 | 0.9654 |
| 08-003 | hPD1ex-H6F | 1.24E−08 | 3.92E+04 | 4.87E−04 | 0.8971 |
| | hCSF1R_His | 1.44E−09 | 1.65E+05 | 2.38E−04 | 0.8795 |
| 08-004 | hPD1ex-H6F | 1.37E−08 | 4.14E+04 | 5.68E−04 | 0.8975 |
| | hCSF1R_His | 2.23E−09 | 1.99E+05 | 4.43E−04 | 0.9104 |
| 08-005 | CD20 peptide | 4.83E−07 | 1.20E+05 | 5.81E−02 | 0.9499 |
| | hCSF1R_His | 1.40E−09 | 1.81E+05 | 2.53E−04 | 0.8549 |
| 08-006 | hCSF1R_His | 1.92E−09 | 1.94E+05 | 3.72E−04 | 0.8812 |
| | CD20 peptide | 2.60E−06 | 1.61E+05 | 4.18E−01 | 0.9840 |

Table 3 shows verification results for nonspecific interaction between the analyte and non-loaded sensors. Nonspecific interaction between analyte and non-loaded sensors was not detected.

TABLE 3
Results of verification of nonspecific interaction
between the analyte and non-loaded sensors

| Sensor type | Analyte | Concentration, nM | Signal, nm |
|---|---|---|---|
| ProA | hPD1ex-H6F | 485.4 | 0.002 |
| ProA | hCSF1R_His | 174.2 | 0.0092 |
| SAX | 08-001 | 2000 | 0.0898 |
| SAX | 08-001 | 500 | 0.0044 |
| SAX | 08-002 | 2000 | 0.0228 |
| SAX | 08-002 | 500 | 0.0123 |
| SAX | 08-005 | 2001 | 0.0311 |
| SAX | 08-005 | 500 | 0.0062 |
| SAX | 08-006 | 2001 | 0.0078 |
| SAX | 08-006 | 500.3 | 0.004 |

From the results provided, all antibody-like molecules 08-001-08-006 demonstrate specific binding to target antigens; further, KID values have the same or similar order of magnitude regardless of the position of the corresponding antigen-binding fragments (as part of Fab fragment or as part of Fab-like fragment comprising β2 microglobulin and modified α3 domain of the CD1b protein instead of CK and CH1 domains).

### Example 5. Analysis of Simultaneous Binding of Antibody-Like Molecules According to the Invention to Two Antigens

Antibody-like molecules 08-003 and 08-004 were analysed for simultaneous binding to two distinct target antigens (hPDlex-H6F and hCSF1R_His) on Forte Bio Octet RED 384. The experiment was carried out on AR2G sensors (Amine Reactive Second-Generation (AR2G) biosensors, ForteBio). The steps of the experiment are shown in Table 4.

TABLE 4
Steps of experiment to verify simultaneous binding of antibody-like
molecules according to the invention to two distinct antigens

| | Experiment | Step | |
|---|---|---|---|
| No. | step | duration (s) | Notes |
| 1 | Baseline | 60 | Auxiliary step, equilibration in water, verification of the |
| | | | signal recorded on sensors |
| 2 | Activation | 300 | Aqueous solution of activators 20 mM EDC (1-(3- |
| | | | Dimethylaminopropyl)-3-ethylcarbodiimide |
| | | | hydrochloride) and 10 mM sNHS (N- |
| | | | hydroxysulfosuccinimide) |
| 3 | Loading | 300 | Loading of hPD1ex-H6F protein (10 μg/ml) in 10 mM |
| | | | sodium acetate buffer solution pH 5.0 |
| 4 | Quenching | 300 | 1M ethanolamine pH 8.5. Quenching of unreacted active |
| | | | groups on sensor surface |
| 5 | Baseline 2 | 300 | Equilibration of sensors in kinetic buffer solution |
| 6 | Loading 2 | 300 | Loading of antibody-like molecule (10 μg/ml) onto sensors |
| | | | is due to interaction with sensor-immobilized hPD1ex-H6F |
| 7 | Baseline 3 | 15 | Equilibration of sensors in kinetic buffer solution, |
| | | | verification of dissociation rate of antibody-like molecule |
| | | | from sensors |
| 8 | Association | 60 | Interaction with hCSF1R_His (10 μg/ml) |
| 9 | Dissociation | 30 | Auxiliary step, confirms the absence of fast dissociation |
| | | | following binding to hCSF1R_His |

Sensors were activated in an aqueous solution comprising 20 mM EDC (1-(3-dimethylaminopropyl)-3-ethylcarbodiimide hydrochloride) and 10 mM sNHS (N-hydroxysulfosuccinimide) for 300 s. The antigen (hPD1ex-H6F) was loaded onto the surface of biosensors in 10 mM sodium acetate buffer, pH 5.0 for 300 seconds. Loading protein concentration was 10 g/ml. Unreacted active centers on the sensor surfaces were quenched in 1M aqueous solution of ethanolamine, pH 8.5 for 300 s. The baseline at the fifth step of the experiment and all subsequent steps were carried out in a kinetic buffer solution (1×KB); volume fraction of added Tween 20 was 0.1%; mass fraction of added BSA was 0.1%; pH 7.4. After recording the baseline (step 5), the antibody-like molecule were loaded onto sensors; the concentration of the antibody-like molecule being loaded was 10 g/ml (step 6). The third baseline was then recorded (step 7). This step demonstrates the absence of fast signal decay, indicating a specific interaction between the loaded antibody-like molecule and hPDlex-H6F immobilized previously onto the sensors. At the next step of association (step 8), the sensors containing immobilized hPD ex-H6F andbound antibody-like molecules were immersed into an hCSF1R_His antigen solution at a concentration of 10 μg/ml. The signal amplification at this step is due to the presence of a FAB fragment to the hCSF1RHis antigen in antibody-like molecules loaded onto the sensors.

Sensograms were analysed using ForteBio Octet Data Analysis 9.0 software. The main results are shown in Table 5. Following the results, samples 08-003 and 08-004 demonstrate simultaneous binding to hPDpex-H6F and hCSFrR His antigens.

TABLE 5
Results of experiment to determine simultaneous interaction of antibody-like molecules
08-003 and 08-004 with two distinct antigens (hPD1ex-H6F and hCSF1R_His).

| | Step 3 | | Step 6 | | Step 8 | | |
|---|---|---|---|---|---|---|---|
| Antibody- | Sensor- | Change | Interaction | Change | Interaction | Change | |
| like | loaded | in signal | with antibody- | in signal | with second | in signal | |
| molecule | proteins | level, nm | like molecule | level, nm | antigen | level, nm | Notes |
| 08-003 | hPD1ex-H6F | 2.1782 | 08-003 | 0.2481 | hCSF1R)_His | 0.0618 | Simultaneously |
| | (10 μg/ml) | | | | (10 μg/ml) | | interacts with |
| | | 2.2164 | 08-003 | 0.205 | 1xKB | −0.0142 | two distinct |
| | | | | | | | antigens |
| | | | | | | | (hPD1ex-H6F |
| | | | | | | | and |
| | | | | | | | hCSF1R_His) |
| 08-004 | hPD1ex-H6F | 2.1893 | 08-004 | 0.2248 | hCSF1R)_His | 0.0722 | Simultaneously |
| | (10 μg/ml) | | | | (10 μg/ml) | | interacts with |
| | | 2.3321 | 08-004 | 0.2654 | 1xKB | −0.0141 | two distinct |
| | | | | | | | antigens |
| | | | | | | | (hPD1ex-H6F |
| | | | | | | | and |
| | | | | | | | hCSF1R_His) |
| Negative | hPD1ex-H6F | 2.256 | 1xKB | −0.0422 | hCSF1R_His | 0.0007 | No non- |
| control | (10 μg/ml) | | | | (10 μg/ml) | | specific |
| | | | | | | | interaction of |
| | | | | | | | the two |
| | | | | | | | antigens with |
| | | | | | | | one another |
| Negative | | 2.1903 | 1xKB | −0.0291 | 1xKB | −0.0051 | No signal |
| control | | | | | | | growth |
| | | | | | | | observed in the |
| | | | | | | | absence of |
| | | | | | | | antibody-like |
| | | | | | | | molecule and |
| | | | | | | | second antigen |

Following the results, antibody-like molecules 08-003 and 08-004 demonstrate simultaneous binding to hPD1ex-H6F and hCSF1R_His antigens.

### Example 6. Analysis of Molecular Weights of Antibody-Like Molecules According to the Invention by Reverse-Phase Ultra-High-Performance Liquid Chromatography (RP UHPLC) with Mass Spectrometric Detection

To confirm the correct assembly of molecules, the molecular weights of the full-length antibody-like molecules according to the invention were analyzed. This method makes it possible to identify full-length antibody-like molecules according to the invention consisting of 4 distinct chains and to distinguish same from by-products formed from another set of chains. These data, along with data from Examples 4 and 5, signify that the technological solution based on substitution of a pair of constant domains CH1-CK with a pair composed of β2 microglobulin and modified α3 domain of the human CD1b protein provides for correct assembly of antibody-like molecules according to the invention.

The analysis was carried out using the Agilent 1290 Infinity II UPLC-Agilent 6530 Q-Tof chromatography-mass spectrometric complex on the BioResolve Polyphenyl RP column (2.1×50 mm, particle diameter 2.7 m; the BioResolve Polyphenyl RP precolumn, 2.1×5 mm, particle diameter 2.7 μm, was also used). Prior to the analysis, all molecules were treated with the PNGase F (Promega) enzyme to remove N-glycans. To this end, the enzyme was diluted with water to a concentration of 1 unit of activity in μl. Sample with a protein content of 50 g was mixed with a buffer solution, a solution of PNGase F was added at the enzyme:protein ratio of 1 unit: 50 μg, the mixture was incubated in a thermostat for 18 hours at (37.0±0.1) ° C.

For analysis, 5 g of the test solution were selected in accordance with the measured protein concentrations and the concentration was adjusted with mobile phase A to 0.2 mg/ml. The sample input volume was 5 μl.

Prior to the analysis, the chromatographic system was equilibrated with a mobile phase at the initial ratio of mobile phases A: 95%, B: 5% (phase A contains 0.1% formic acid solution in water, phase B contains 0.1% formic acid solution, 30% acetonitrile in isopropyl alcohol) for at least 30 min until stable pressure was achieved. The mass spectrometer was calibrated using a calibration standard in accordance with the manufacturer's guidelines.

Sample was separated at a flow rate of 0.45 ml/min in concentration gradient of mobile phase B (5% to 30% from 2 to 3 minutes following sample introduction, then 30% to 35% from 3 to 8 minutes, then 35 to 95% from 8 to 9 minutes) at a column temperature of (60±1) ° C.; chromatogram was obtained at a wavelength of 280 nm.

Data was processed in Protein Metrics.

Results of the mass analysis for antibody-like molecules according to the invention are shown in Table 6. In the table, each antibody-like molecule is provided with the percentage of masses corresponding to a correctly assembled antibody-like molecule consisting of four distinct chains relative to all masses determined by the instrument.

TABLE 6
Content of correctly and incorrectly assembled antibody-
like molecules in samples treated with PNGase F enzyme

| | Sample | | | | | |
|---|---|---|---|---|---|---|
| | 08- | 08- | 08- | 08- | 08- | 08- |
| Assembly variants | 001 | 002 | 003 | 004 | 005 | 006 |
| Correctly assembled | 94.4 | 97.4 | 97.8 | 88.3 | 100 | 91.3 |
| antibody-like molecule, % | | | | | | |
| incorrectly assembled | 5.6 | 2.6 | 2.2 | 11.7 | — | 8.8 |
| antibody-like molecule, % | | | | | | |

The results of the analysis allow to conclude that the mass of full-sized antibody-like molecules consisting of four distinct chains is predominant in all samples.

### Example 7. Confirmation of Correct Assembly of Antibody-Like Molecules According to the Invention Using Proteolysis and Mass Analysis of Resulting Fragments

To directly check for correct pairing of light and heavy chains, the antibody-like molecules according to the invention were cleaved by the GingisKHAN protease (Genovis) whose recognition site is located in the hinge region of antibody-like molecule ( . . . KSCDK/THTCPPCP . . . ). Such proteolysis results in breaking down of the full-length antibody-like molecule into an Fc fragment, Fab fragment, and Fab-like fragment comprising β2 microglobulin and modified α3 domain of the human CD1b protein substituting CK and CH1 domains. Fragments of the antibody-like molecule were analyzed following proteolysis using vertical electrophoresis under non-reducing conditions; also, the resulting fragments were subjected to mass spectrometry.

The proteolysis reaction mixture contained 40 μg of the antibody-like molecule and 40 units of the GingisKHAN enzyme (at the ratio of 1 enzyme unit: 1 μg of protein) in a buffer composed of 100 mM Tris-HCl, pH 8.0, 1 mM cysteine in a volume of 60 μl. The reaction was carried out in a thermostat at (37.0±0.1) ° C. for 1 hour and stopped by adding iodoacetamide to a concentration of 10 mM. For electrophoresis, a buffer containing SDS (up to a concentration of 1% SDS) was added to the resulting samples, and SDS gel electrophoresis was performed under non-reducing conditions.

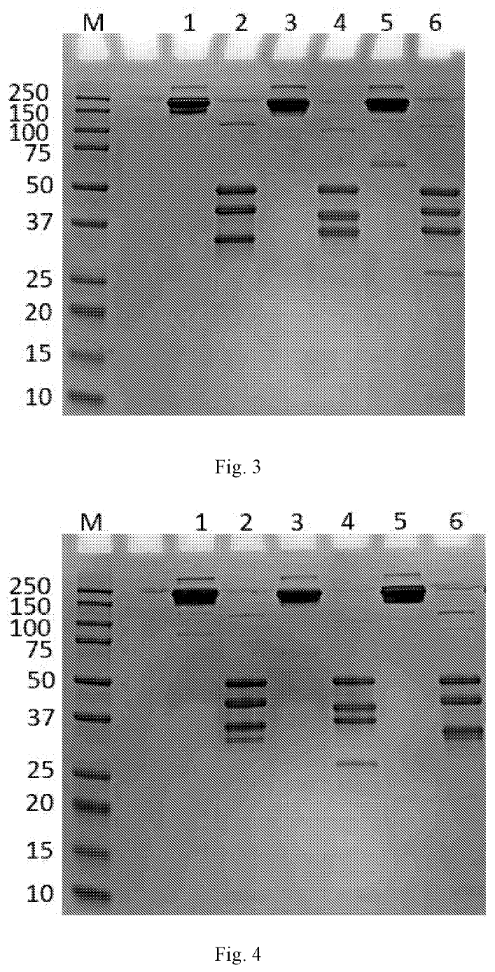

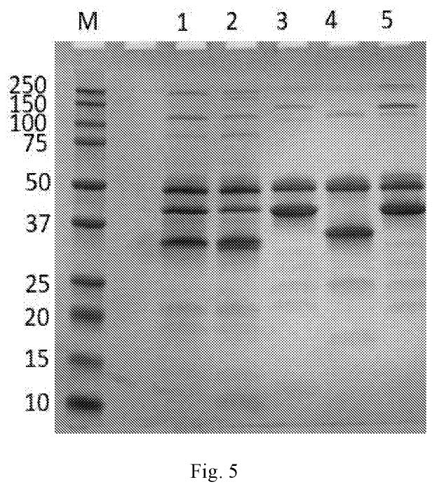

FIGS. 3-5 show the results of SDS gel electrophoresis. Following treatment of the antibody-like molecules (08-001-08-006) with the GingisKHAN protease (FIGS. 3 and 4), 3 major fragments were formed, which were observed in electrophoregram in 40-50 kDa region and showed distinct mobilities in PAAG. A monospecific molecule comprising a dimerization unit of membrane-proximal domains of the human CD1b protein and comprising variable fragments of antibody Prolgolimab (FIG. 5, lane 4), as well as monospecific antibodies in the IgG1 format comprising variable fragments of antibody Prolgolimab and of antibody Ocrelizumab (FIG. 5, lanes 3, 5) were also treated with the GingisKHAN protease. As a result of comparing the electrophoretic mobilities of fragments formed during proteolysis of monospecific and bispecific antibody-like molecules, it was concluded that the fragment showing the lowest mobility level corresponds to Fc fragment, that showing medium mobility level corresponds to Fab fragment, and the fragment showing the highest mobility level corresponds to Fab-like fragment comprising β2 microglobulin and modified α3 domain of the CD1b protein substituting CK and CH1 domains. In the case of antibody-like molecules 08-003-08-006, the electrophoregram showed a further fragment formed as a result of nonspecific cleavage of the variable domain of anti-CSF1R fragment. In order to determine the exact masses of the fragments formed as a result of proteolysis, we conducted mass spectrometry analysis.

Prior to the mass spectrometry analysis, the samples, immediately following adding iodoacetamide, were transferred to 50 mM ammonium bicarbonate, pH (7.6±0.2), on Zeba (MWCO 7 kDa) columns according to the manufacturer's guidelines; thereafter, PNGase F at the ratio of 1 enzyme unit: 50 g of protein was added to the samples and the samples were thermostated for 18 hours at (37.0±0.1) ° C.

Mass analysis of the resulting fragments was carried out as described in Example 6 with the following changes: to achieve a better peak resolution, the sample was separated at a flow rate of 0.4 ml/min in a concentration gradient of mobile phase B (5% to 55% from 2 to 20 minutes following sample introduction, then 55% to 95% from 20 to 21 minutes) at column temperature (60±1° C.; phase A comprised 0.1% formic acid, 0.02% trifluoroacetic acid in water, phase B comprised 0.1% formic acid solution, 30% acetonitrile in isopropyl alcohol.

Results of RP UHPLC with mass spectrometric detection are shown in Table 8.

TABLE 8
Content of fragments in samples treated
with GingisKHAN and PNGase F enzymes

| | Sample | | | | | |
|---|---|---|---|---|---|---|
| | 08- | 08- | 08- | 08- | 08- | 08- |
| Fragment content, % | 001 | 002 | 003 | 004 | 005 | 006 |
| Correctly assembled | 32.9 | 38.2 | 32.8 | 47.0 | 45.3 | 33.6 |
| Fab fragment | | | | | | |
| Correctly assembled | 46.0 | 44.6 | 39.9 | 26.3 | 29.0 | 38.1 |
| Fab-like fragment | | | | | | |
| Correctly assembled | 19.2 | 16.1 | 21.8 | 15.4 | 19.3 | 17.3 |
| Fc-hole and Fc-knob | | | | | | |
| dimers | | | | | | |
| Fc-hole homodimer | 0.5 | 0.5 | 0.3 | 0.4 | 0.4 | 0.4 |
| incorrectly | 0.7 | — | — | 0.1 | — | 1.7 |
| assembled Fab-like | | | | | | |
| fragments | | | | | | |

Unidentified impurities

| | | | | | | |
|---|---|---|---|---|---|---|
| 36043 Da | — | — | — | 3.2 | — | 3.2 |
| 36060 Da | — | — | — | 7.2 | — | 5.4 |
| 37076 Da | — | — | 4.5 | — | 5.4 | — |
| 49471 Da | 0.3 | 0.3 | 0.3 | 0.2 | 0.2 | 0.2 |
| 49600 Da | 0.4 | 0.4 | 0.4 | 0.2 | 0.4 | 0.2 |

The results show that all antibody-like molecules according to the invention broke down following proteolysis into fragments corresponding in mass to an Fc fragment, Fab fragment and Fab-like fragment comprising β2 microglobulin and modified 03 domain of the human CD1b protein substituting CK and CH1 domains, signifying that the technological solution based on the substitution of the pair of constant domains CH1-CK with a pair composed of β2 microglobulin and modified 03 domain of the human CD1b protein provides for correct assembly of the antibody-like molecules according to the invention.

1. An antibody-like molecule that specifically binds to first and second targets, wherein said antibody-like molecule comprises:
1) a first antigen-binding fragment that specifically binds to the first target and comprises:
a) the light chain of the first antigen-binding fragment, which light chain comprises a light chain variable domain and a light chain constant domain;
and
b) the heavy chain of the first antigen-binding fragment, which heavy chain comprises a heavy chain variable domain and heavy chain constant domains of antibody that include a first (CH1) heavy chain constant domain and an Fc fragment monomer comprising second (CH2) and third (CH3) heavy chain constant domains;
and
2) a second antigen-binding fragment that specifically binds to the second target and comprises:
(i) a) the light chain of the second antigen-binding fragment, which light chain comprises a light chain variable domain and a constant domain that is β2 microglobulin (β2M) of human CD1b (cluster of differentiation 1) protein with the amino acid sequence of SEQ ID NO: 1;
and
b) the heavy chain of the second antigen-binding fragment, which heavy chain comprises a heavy chain variable domain, a constant domain that is the α3 membrane-proximal domain of the human CD1b protein with the amino acid sequence of SEQ ID NO: 2, and an Fc fragment monomer comprising second (CH2) and third (CH3) heavy chain constant domains;
or
(ii) a) the light chain of the second antigen-binding fragment, which light chain comprises a light chain variable domain and a constant domain that is the α3 membrane-proximal domain of the human CD1b protein with the amino acid sequence of SEQ ID NO: 2;
and
b) the heavy chain of the second antigen-binding fragment, which heavy chain comprises a heavy chain variable domain, a constant domain that is β2 microglobulin (β2M) of the human CD1b protein with the amino acid sequence of SEQ ID NO: 1, and an Fc fragment monomer comprising second (CH2) and third (CH3) heavy chain constant domains;
wherein the β2 microglobulin (β2M) of the human CD1b protein with the amino acid sequence of SEQ ID NO: 1 and the α3 membrane-proximal domain of the human CD1b protein with the amino acid sequence of SEQ ID NO: 2 form therebetween a heterodimer that is stabilized by a disulfide bond.

2. The antibody-like molecule according to claim 1, wherein the CH3 domain of one heavy chain and the CH3 domain of other heavy chain contact one another via surfaces that are modified to form the antibody-like molecule, wherein such modifications in the CH3 domains of heavy chains are substitutions to provide for heterodimerization.

3. The antibody-like molecule according to claim 2, wherein
a) the CH3 domain of one heavy chain is modified so that on the surface ofthe CH3 domain of one heavy chain contacting the surface of the CH3 domain of other heavy chain in the antibody-like molecule, the amino acid residue is substituted with an amino acid residue that has a larger side chain volume, leading to formation of a knob on the surface of the CH3 domain of one heavy chain that can fit into a hole on the surface of the CH3 domain of other heavy chain,
and
b) the CH3 domain of other heavy chain is modified so that on the surface of the CH3 domain of the second heavy chain contacting the surface of the CH3 domain of the first heavy chain in the antibody-like molecule, the amino acid residue is substituted with an amino acid residue that has a smaller side chain volume, leading to formation of a hole on the surface of the CH3 domain of the second heavy chain capable of fitting a knob on the interface of the CH3 domain of the first heavy chain;
wherein said amino acid residue that has a larger side chain volume is selected from a group including arginine (R), phenylalanine (F), tyrosine (Y), tryptophan (W),
and wherein said amino acid residue that has a smaller side chain volume is selected from a group including alanine (A), serine (S), threonine (T), valine (V).

4. The antibody-like molecule according to claim 1, wherein the first light chain constant domain of the antibody-like molecule is selected from CK or CL.

5. The antibody-like molecule according to claim 1, wherein the CH3 domains of the antibody-like molecule are modified by way of introduction of cysteine (C) as an amino acid into the corresponding positions of each CH3 domain so that a disulfide bridge may form between the both CH3 domains.

6. The antibody-like molecule according to any one of claims 2-3, wherein the CH3 domain of one heavy chain is modified to form Knob, and the CH3 domain of other heavy chain is modified to form Hole, or vice versa.

7. The antibody-like molecule according to claim 6, wherein the CH3 domain of one heavy chain has S354C/T366W amino acid substitutions according to the EU numbering scheme for amino acids of antibodies, and the CH3 domain of other heavy chain has Y349C/T366S/L368A/Y407V amino acid substitutions according to the EU numbering scheme for amino acids of antibodies.

8. The antibody-like molecule according to claim 6, wherein the CH3 domain of one heavy chain has Y349C/T366S/L368A/Y407 amino acid substitutions according to the EU numbering scheme for amino acids of antibodies, and the CH3 domain of other heavy chain has S354C/T366W amino acid substitutions according to the EU numbering scheme for amino acids of antibodies.

9. The antibody-like molecule according to claim 1, wherein the Fc fragment belongs to IgG.

10. The antibody-like molecule according to claim 9, wherein the Fc fragment isotype is selected from the group: human IgG1, IgG2, or IgG4.

11. The antibody-like molecule according to claim 1, wherein the Fc fragment monomer comprises substitutions leading to no ADCC, CDC and/or ADCP properties in the antibody-like molecule.

12. The antibody-like molecule according to claim 11, wherein the Fc fragment monomer comprises L234A and L235A substitutions according to the EU numbering scheme for amino acids of antibodies.

13. The antibody-like molecule according to claim 1, wherein the Fc fragment monomer comprises substitutions leading to prolonged action of the antibody-like molecule.

14. The antibody-like molecule according to claim 13, wherein the Fc fragment monomer comprises M252Y, S254T and T256E substitutions according to the EU numbering scheme for amino acids of antibodies.

15. The antibody-like molecule according to claim 1, wherein the Fc fragment monomer comprises substitutions leading to enhanced ADCC, CDC and/or ADCP properties in the antibody-like molecule.

16. The antibody-like molecule according to claim 15, wherein the Fc fragment monomer comprises E345R substitution according to the EU numbering scheme for amino acids of antibodies.

17. The antibody-like molecule according to any one of claims 1-16, wherein the first and second targets may each be independently selected from a group comprising: CD20, BCMA, PD-1, PD-L1, CD47, GD2, AXL, TGF beta, CSF1R, blood clotting factor 9 (FIX), blood clotting factor 10 (FX), TNF alfa, IL17A, IL17F or CD3.

18. The antibody-like molecule according to any one of claims 1-16, wherein the antibody-like molecule is a multispecific antibody-like molecule.

19. The antibody-like molecule according to any one of claims 1-16, wherein the antibody-like molecule is a bispecific, trispecific, tetraspecific, pentaspecific, hexaspecific, heptaspecific, octaspecific or nonaspecific antibody-like molecule.

20. The antibody-like molecule according to any one of claims 1-16, wherein the antibody-like molecule is a bivalent bispecific antibody-like molecule.

21. An isolated nucleic acid that encodes the antibody-like molecule according to any one of claims 1-20.

22. The isolated nucleic acid according to claim 21, wherein the nucleic acid is DNA.

23. An expression vector comprising the nucleic acid according to any one of claims 21-22.

24. A method for producing a host cell to produce the antibody-like molecule according to any one of claims 1-20, comprising cell transformation by the vector according to claim 23.

25. A host cell for producing the antibody-like molecule according to any one of claims 1-20, comprising the nucleic acid according to claims 21-22.

26. A method for producing the antibody-like molecule according to claims 1-20, wherein the method comprises the following steps of:
a) transforming a host cell
with expression vectors comprising nucleic acid molecules encoding the light and heavy chains of the first antigen-binding fragment of the antibody-like molecule,
with expression vectors comprising nucleic acid molecules encoding the light and heavy chains of the second antigen-binding fragment of the antibody-like molecule,
b) culturing the host cell under conditions suitable for synthesis of said antibody-like molecule; and
c) isolating said antibody-like molecule from cell culture.


SEQUENCE LISTING (33 sequences)

SEQ ID NO: 1 - protein, 98 aa, Homo sapiens
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRD

SEQ ID NO: 2 - protein, 100 aa, synthetic construct
QRQVKPEAWLSKGPSPGPGRLQLVCHVSGFYPKPVWVMWMRGEQEQQGTQLGDILPNADWTWYLRATLDVADGEAAGLSCRVKHSSLEGQDIILYWRGSC

SEQ ID NO: 3 - protein, 97 aa, Homo sapiens
QRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWR

SEQ ID NO: 4 - protein, 129 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSS

SEQ ID NO: 5 - protein, 107 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQ

SEQ ID NO: 6 - protein, 122 aa, synthetic construct [sequence omitted]

SEQ ID NO: 7 - protein, 106 aa, synthetic construct [sequence omitted]

SEQ ID NO: 8 - protein, 124 aa, synthetic construct
QVQLVESGGGLVRPGGSLRLSCAASEFTFDDYAMSWVRQAPGKGLEWVSAISWNGGSTNYADSVKGRFTISRDNAKNSLYLQMNSLRAEDTALYYCASTVEVAHRRLYKYLEVWGQGTLVTVSS

SEQ ID NO: 9 - protein, 108 aa, synthetic construct
QAGLTQPPSVSVAPGQTARITCGGNNIGSKSVHWYQQKPGQAPVLVVYGDSDRPSGIPERFSGSNSGNTATLTISRVEAGDEADYYCQVWDISSDHVVFGGGTQLTVL

SEQ ID NO: 10 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 206 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKQRQVKPEAWLSKGPSPGPGRLQLVCHVSGFYPKPVWVMWMRGEQEQQGTQLGDILPNADWTWYLRATLDVADGEAAGLSCRVKHSSLEGQDIILYWRGSC

SEQ ID NO: 14 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 207 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQQRQVKPEAWLSKGPSPGPGRLQLVCHVSGFYPKPVWVMWMRGEQEQQGTQLGDILPNADWTWYLRATLDVADGEAAGLSCRVKHSSLEGQDIILYWRGSC

SEQ ID NO: 18 - protein, 454 aa, synthetic construct
QVQLVESGGGLVRPGGSLRLSCAASEFTFDDYAMSWVRQAPGKGLEWVSAISWNGGSTNYADSVKGRFTISRDNAKNSLYLQMNSLRAEDTALYYCASTVEVAHRRLYKYLEVWGQGTLVTVSSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 208 aa, synthetic construct
QAGLTQPPSVSVAPGQTARITCGGNNIGSKSVHWYQQKPGQAPVLVVYGDSDRPSGIPERFSGSNSGNTATLTISRVEAGDEADYYCQVWDISSDHVVFGGGTQLTVLQRQVKPEAWLSKGPSPGPGRLQLVCHVSGFYPKPVWVMWMRGEQEQQGTQLGDILPNADWTWYLRATLDVADGEAAGLSCRVKHSSLEGQDIILYWRGSC

SEQ ID NO: 20 - protein, 454 aa, synthetic construct
QVQLVESGGGLVRPGGSLRLSCAASEFTFDDYAMSWVRQAPGKGLEWVSAISWNGGSTNYADSVKGRFTISRDNAKNSLYLQMNSLRAEDTALYYCASTVEVAHRRLYKYLEVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 215 aa, synthetic construct
QAGLTQPPSVSVAPGQTARITCGGNNIGSKSVHWYQQKPGQAPVLVVYGDSDRPSGIPERFSGSNSGNTATLTISRVEAGDEADYYCQVWDISSDHVVFGGGTQLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 1377 bp, synthetic construct
  source 1..1377: mol_type = other DNA
caggttcagctggtgcagtctgggggaggcttggtgcagcctggggggtctctgagactctcctgtgcagcctctggattcaccttcagtagctactggatgtattgggtccgccaggttccagggaaggggctcgagtgggtctcagctattgatactggtggtggtaggacatactatgcagactccgtgaagggccggttcgccatctccagagtcaacgccaagaacacaatgtatctgcaaatgaacagcctgagagccgaggacacggccgtgtattactgtgcgagagatgaaggcgggggtacgggttggggtgttttgaaggattggccctacggcttggacgcatggggccaggggaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcagggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 23 - DNA, 642 bp, synthetic construct
  source 1..642: mol_type = other DNA
cagcctgtgctgactcagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactattactgtcaggtgtgggacagcagcactgctgtcttcggaactgggaccaagctgaccgtcctacagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 24 - DNA, 1356 bp, synthetic construct
  source 1..1356: mol_type = other DNA
gaagtccaattagtagaatcaggcggcggcttagtgcaacccggtggctcactgcgtttgagctgtgctgctagtggctatacctttacgtcctacaacatgcattgggtgagacaagcacctggcaagggtctcgaatgggtaggcgctatctatccaggaaatggtgacacctcttacaaccagaagtttaagggtcgctttaccatctcggtcgataagagcaagaacaccctgtacctacagatgaattcgttacgggcagaggacacagccgtctattactgcgctcgggtggtatattactccaactcttactggtacttcgacgtttggggccagggaactcttgtgaccgtgagctctatccaacgcacaccaaagatccaggtctattcgcggcatccggcagaaaacggaaagagtaacttcctgaactgctatgtatcgggctttcatccaagtgatatcgaggtggacctgctgaagaacggagagagaatcgagaaggtggaacactcggacctgagcttctccaaggactggagtttctacctgctgtactataccgagttcaccccgacagaaaaggacgaatacgcatgtcgggtcaatcatgtcaccctgagtcagccgaaaatcgtaaagtgggatcgggacgagcccaaatcttgtgacaaaactcacacatgcccaccgtgcccagcacctgaagccgctgggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 25 - DNA, 618 bp, synthetic construct
  source 1..618: mol_type = other DNA
gacattcaaatgacccaatcgccatcttcgctttcagcttcggtaggagatcgcgtaaccattacatgccgggcctcgtcttcggtctcgtacatgcattggtatcagcaaaaaccaggaaaagctcccaaacccctgatctacgcaccctctaacctggcttctggcgtcccgtcgaggttttcgggttctggctctggcacagactttacacttaccatctcgtcgctgcagccagaggattttgccacctactactgtcagcaatggtcttttaatccaccaacattcggccaggggaccaaagtagaaatcaaacagagacaggttaaaccagaggcatggctaagtaagggaccttcacctggacctggtagattacaattagtatgtcatgtgtcgggattttatcctaaacctgtttgggtcatgtggatgaggggcgagcaagagcagcaaggaacacagttaggcgacatcctgccgaatgcagactggacatggtacttaagagcaaccctggatgtagcggacggcgaagccgctggattatcgtgccgggtaaagcattcgtcgctggagggccaagacataatactctattggcgcggtagttgc

SEQ ID NO: 26 - DNA, 1356 bp, synthetic construct
  source 1..1356: mol_type = other DNA
gaagtccaattagtagaatcaggcggcggcttagtgcaacccggtggctcactgcgtttgagctgtgctgctagtggctatacctttacgtcctacaacatgcattgggtgagacaagcacctggcaagggtctcgaatgggtaggcgctatctatccaggaaatggtgacacctcttacaaccagaagtttaagggtcgctttaccatctcggtcgataagagcaagaacaccctgtacctacagatgaattcgttacgggcagaggacacagccgtctattactgcgctcgggtggtatattactccaactcttactggtacttcgacgtttggggccagggaactcttgtgaccgtgagctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcagggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 27 - DNA, 639 bp, synthetic construct
  source 1..639: mol_type = other DNA
gacattcaaatgacccaatcgccatcttcgctttcagcttcggtaggagatcgcgtaaccattacatgccgggcctcgtcttcggtctcgtacatgcattggtatcagcaaaaaccaggaaaagctcccaaacccctgatctacgcaccctctaacctggcttctggcgtcccgtcgaggttttcgggttctggctctggcacagactttacacttaccatctcgtcgctgcagccagaggattttgccacctactactgtcagcaatggtcttttaatccaccaacattcggccaggggaccaaagtagaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28 - DNA, 1377 bp, synthetic construct
  source 1..1377: mol_type = other DNA
caggttcagctggtgcagtctgggggaggcttggtgcagcctggggggtctctgagactctcctgtgcagcctctggattcaccttcagtagctactggatgtattgggtccgccaggttccagggaaggggctcgagtgggtctcagctattgatactggtggtggtaggacatactatgcagactccgtgaagggccggttcgccatctccagagtcaacgccaagaacacaatgtatctgcaaatgaacagcctgagagccgaggacacggccgtgtattactgtgcgagagatgaaggcgggggtacgggttggggtgttttgaaggattggccctacggcttggacgcatggggccaggggaccctggtcaccgtctcctcaatccaacgcacaccaaagatccaggtctattcgcggcatccggcagaaaacggaaagagtaacttcctgaactgctatgtatcgggctttcatccaagtgatatcgaggtggacctgctgaagaacggagagagaatcgagaaggtggaacactcggacctgagcttctccaaggactggagtttctacctgctgtactataccgagttcaccccgacagaaaaggacgaatacgcatgtcgggtcaatcatgtcaccctgagtcagccgaaaatcgtaaagtgggatcgggacgagcccaaatcttgtgacaaaactcacacatgcccaccgtgcccagcacctgaagccgctgggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 29 - DNA, 621 bp, synthetic construct
  source 1..621: mol_type = other DNA
cagcctgtgctgactcagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactattactgtcaggtgtgggacagcagcactgctgtcttcggaactgggaccaagctgaccgtcctacagcagagacaggttaaaccagaggcatggctaagtaagggaccttcacctggacctggtagattacaattagtatgtcatgtgtcgggattttatcctaaacctgtttgggtcatgtggatgaggggcgagcaagagcagcaaggaacacagttaggcgacatcctgccgaatgcagactggacatggtacttaagagcaaccctggatgtagcggacggcgaagccgctggattatcgtgccgggtaaagcattcgtcgctggagggccaagacataatactctattggcgcggtagttgc

SEQ ID NO: 30 - DNA, 1362 bp, synthetic construct
  source 1..1362: mol_type = other DNA
caagtccagttggtagaaagcggcggaggtttggttaggcccggaggcagcctgagattatcctgtgcagcaagcgagttcacttttgatgactatgcgatgagttgggtgcgacaagctcctggaaagggattggaatgggtgtcagcgatctcatggaatggcggatcaactaactacgcagattctgtcaagggcaggtttaccatctcccgggataacgcaaagaatagtctgtaccttcaaatgaacagtctgcgcgctgaggacactgccttatattattgcgcaagcactgtggaagtagcccaccgacggttgtataagtacctcgaagtatggggtcagggaacactggtcacagtaagctcaatccaacgcacaccaaagatccaggtctattcgcggcatccggcagaaaacggaaagagtaacttcctgaactgctatgtatcgggctttcatccaagtgatatcgaggtggacctgctgaagaacggagagagaatcgagaaggtggaacactcggacctgagcttctccaaggactggagtttctacctgctgtactataccgagttcaccccgacagaaaaggacgaatacgcatgtcgggtcaatcatgtcaccctgagtcagccgaaaatcgtaaagtgggatcgggacgagcccaaatcttgtgacaaaactcacacatgcccaccgtgcccagcacctgaagccgctgggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 31 - DNA, 624 bp, synthetic construct
  source 1..624: mol_type = other DNA
caggcagggctgactcagccaccctcggtgtcagtggccccaggacagacggccaggattacctgtgggggaaacaacattggaagtaaaagtgtgcactggtaccagcagaagccaggccaggcccctgtgctggtcgtctatggtgatagcgaccggccctcagggatccctgagcgattctctggctccaactctgggaacacggccaccctgaccatcagcagggtcgaagccggggatgaggccgactattactgtcaggtgtgggatattagtagtgatcatgtggtattcggcggagggacccagctcaccgttttacagagacaggttaaaccagaggcatggctaagtaagggaccttcacctggacctggtagattacaattagtatgtcatgtgtcgggattttatcctaaacctgtttgggtcatgtggatgaggggcgagcaagagcagcaaggaacacagttaggcgacatcctgccgaatgcagactggacatggtacttaagagcaaccctggatgtagcggacggcgaagccgctggattatcgtgccgggtaaagcattcgtcgctggagggccaagacataatactctattggcgcggtagttgc

SEQ ID NO: 32 - DNA, 1362 bp, synthetic construct
  source 1..1362: mol_type = other DNA
caagtccagttggtagaaagcggcggaggtttggttaggcccggaggcagcctgagattatcctgtgcagcaagcgagttcacttttgatgactatgcgatgagttgggtgcgacaagctcctggaaagggattggaatgggtgtcagcgatctcatggaatggcggatcaactaactacgcagattctgtcaagggcaggtttaccatctcccgggataacgcaaagaatagtctgtaccttcaaatgaacagtctgcgcgctgaggacactgccttatattattgcgcaagcactgtggaagtagcccaccgacggttgtataagtacctcgaagtatggggtcagggaacactggtcacagtaagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcagggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 33 - DNA, 645 bp, synthetic construct
  source 1..645: mol_type = other DNA
caggcagggctgactcagccaccctcggtgtcagtggccccaggacagacggccaggattacctgtgggggaaacaacattggaagtaaaagtgtgcactggtaccagcagaagccaggccaggcccctgtgctggtcgtctatggtgatagcgaccggccctcagggatccctgagcgattctctggctccaactctgggaacacggccaccctgaccatcagcagggtcgaagccggggatgaggccgactattactgtcaggtgtgggatattagtagtgatcatgtggtattcggcggagggacccagctcaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt